(19) United States
(12) Patent Application Publication
(10) Pub. No.: US 2023/0280322 A1
(43) Pub. Date: Sep. 7, 2023

(54) HYDROGEN GAS SENSOR AND METHODS AND SYSTEMS USING SAME TO QUANTITATE HYDROGEN GAS AND/OR TO ASSESS HYDROGEN GAS PURITY

(71) Applicant: Giner, Inc., Newton, MA (US)

(72) Inventors: Badawi M. Dweik, Foxboro, MA (US); William R. McInerney, Newton, MA (US)

(21) Appl. No.: 18/112,881

(22) Filed: Feb. 22, 2023

Related U.S. Application Data
(60) Provisional application No. 63/312,445, filed on Feb. 22, 2022.

(51) Int. Cl. G01N 33/00; G01N 27/22

(52) U.S. Cl. CPC G01N 33/005; G01N 27/226; G01N 33/0073; G01N 33/0062; G01N 2027/222

(57) ABSTRACT

Hydrogen gas sensor and methods and systems using same to quantitate hydrogen gas and/or assess hydrogen gas purity. In one embodiment, the hydrogen gas sensor may include a planar, electrically non-conductive substrate. A working electrode, a reference electrode, a first counter electrode, and a second counter electrode may be positioned on a top surface of the substrate. The working electrode and the second counter electrode may be made of platinum, the first counter electrode may be made of ruthenium oxide, and the reference electrode may be made of silver chloride. The first counter electrode may have a surface area considerably greater than that of the working electrode. A proton exchange membrane may be deposited over the working electrode, the reference electrode, and the first and second counter electrodes. The electrodes and proton exchange membrane may be enclosed within a housing having an aperture to allow gas to enter for analysis.

### CROSS-REFERENCE TO RELATED APPLICATIONS

The present application claims the benefit under 35 U.S.C. 119(e) of U.S. Provisional Patent Application No. 63/312,445, inventors Badawi M. Dweik et al., filed Feb. 22, 2022, the disclosure of which is incorporated herein by reference in its entirety.

### BACKGROUND OF THE INVENTION

The present invention relates generally to hydrogen gas sensors and relates more particularly to a novel hydrogen gas sensor and to methods and systems using the same to quantitate hydrogen gas and/or to assess hydrogen gas purity.

There is currently a great need for green energy production. As a result, hydrogen-based technologies, which have the abundant potential for providing green energy, have received considerable attention. However, to realize a functional hydrogen-based economy, it is necessary not only for improvements to be made with respect to systems and processes that involve hydrogen gas (e.g., fuel cells, electrolyzers, liquefaction, etc.) but also for many other ancillary technologies to be developed to mitigate crucial problems.

For example, a key technology that must be advanced to meet the needs of a hydrogen-based economy is the field of hydrogen gas sensors. At present, there are many types of hydrogen gas sensors that exist commercially that can detect hydrogen gas at parts per million (ppm) levels or higher using sensors that employ electrochemical, thermal, or optical technologies. These types of sensors are critical in addressing issues, such as detecting hydrogen concentration in process streams and monitoring environments for safety. Unfortunately, however, there are still key measuring conditions that are not commercially attainable using current hydrogen gas sensor technology. Specifically, trace level detection of hydrogen gas at parts per billion (ppb) levels is not commercially viable with current technology. This is because most commercial sensors are either not sensitive at ppb levels or, alternatively, are sufficiently sensitive at ppb levels but are not practical as cost-effective, field-ready sensors. The capability of measuring hydrogen losses at very low levels is critical because hydrogen is the lightest molecule and rapidly diffuses and rises. As a result, given its inherent properties, hydrogen gas is highly susceptible to leaking out of fixtures, which not only is bad in terms of financial losses but also has a detrimental effect on the environment. One of the key motivations for switching to a hydrogen-based economy is that hydrogen gas can be a greener source of energy. However, this is not true in practice if hydrogen gas leaks are not managed appropriately. In fact, hydrogen gas has a 100-time more pronounced global warming effect than carbon dioxide. (See Ocko et al., “Climate Consequences of Hydrogen Leakage,” Atmos. Chem. Phys. Discuss. (2022), which is incorporated herein by reference.) Consequently, even if all fossil fuels are replaced with hydrogen gas but leaks are not properly managed, global warming effects could be twice as bad as they would otherwise be. By contrast, minimal leaking of hydrogen gas could result in an 80% reduction in global warming effects.

One well-known type of gas sensor, which may be used to detect carbon monoxide, hydrogen gas, and other easily oxidizable or reducible gases and vapors, is disclosed in U.S. Pat. No. 4,820,386, inventors LaConti et al., which issued Apr. 11, 1989, and which is incorporated herein by reference. In particular, according to the aforementioned patent, there is disclosed a fast response diffusion-type sensor cell that comprises a three-electrode hydrated proton-conducting membrane cell configuration, with all electrodes in intimate contact with the same proton-conducting membrane. This system, which is liquid electrolyte-free, has a porous gas-diffusion sensing electrode and a counter electrode located on the same side of and in intimate contact with the proton-conducting membrane. The reference electrode is spatially located on the same or opposite side of the membrane as the sensing and counter electrodes. The cell configuration is said to be advantageous in that (1) the ionic resistance value between the sensing/reference electrodes is lower than that between the sensing/counter electrodes, and (2) the sensing and counter electrodes are on the same side of the membrane and connected by one or more hydrated proton-exchange membrane channels leads to faster response times and greater immunity to interference from counter electrode reaction products.

A hydrogen gas sensor of the type described in the foregoing patent can operate at ppm levels of detection and is designed in a manner that is manufacturable and cost-effective. However, such a sensor is not practical for lower sensitivity requirements, such as measuring at ppb level concentrations of hydrogen gas. Additionally, over time, water from an associated reservoir used to hydrate the membrane may become depleted, and the membrane can dry up. Consequently, a key issue in improving such sensors is increasing sensitivity and mitigating issues related to membrane hydration.

In addition to hydrogen gas quantitation, another key measurement parameter relating to hydrogen-based technologies is hydrogen gas purity. For example, when supplying hydrogen gas to a fuel cell, it is critical to ensure that there are no interfering species, such as carbon monoxide (CO), ammonia (NH₃), or hydrogen sulfide (H₂S), that are admixed with the hydrogen gas and that can lower the performance, or even irreversibly damage, the catalyst of the fuel cell. Hydrogen gas purity must be assessed by investigating the presence of all common potentially interfering species; consequently, assessing purity can be complicated since most sensors only target one particular type of interfering species. Cost-effective sensors for water and oxygen can be used inline at low detection limits at a reasonable cost; however, sensors for other interfering gas species are more expensive. (See Arrhenius et al., “Detection of Contaminants in Hydrogen Fuel for Fuel Cell Electrical Vehicles with Sensors—Available Technology, Testing Protocols and Implementation Challenges,” Processes, 10(1):20 (2022), which is incorporated herein by reference.) Because existing sensor suites are too expensive to use inline for all relevant gas species, hydrogen gas purity is typically assessed by periodic off-site laboratory analysis. A measurement technique that can be used to measure a broad range of relevant interfering species in a field setting would be ideal; however, no such technique currently exists at a commercial scale.

An example of a sensor that is designed to measure hydrogen gas purity is disclosed in U.S. Pat. No. 10,490,833 B1, inventors Brosha et al., which was issued Nov. 26, 2019, and which is incorporated herein by reference. According to the aforementioned patent, there is disclosed a fuel quality analyzer for detecting contaminants in a fuel supply, the analyzer including an anode flow field plate defining a first fuel flow field channel and a fuel inlet port, a cathode flow field plate defining a second fuel flow field channel and a fuel outlet port, a polymer electrolyte membrane between the anode and cathode flow field plates, a first electrode between the anode flow field plate and the polymer electrolyte membrane, and a second electrode between the cathode flow field plate and the polymer electrolyte membrane. The second electrode has a higher platinum loading than the first electrode. A reservoir volume is defined by the anode and cathode flow field plates. At least a portion of the polymer electrolyte membrane extends into the reservoir volume. The reservoir volume is configured to retain water to humidify the polymer electrolyte membrane.

The analyzer device of the foregoing patent is operated by flowing hydrogen gas into the anode flow field. This causes the hydrogen gas to be oxidized to protons. The protons are then conducted across the membrane to the cathode, where they are reduced back to hydrogen gas. The overall cell current obtained during this process is used for measurement. A baseline current is reached during steady-state operation in a pure hydrogen environment. Drops in current are detected and are related to the presence of interferent species. The foregoing analyzer device uses a broad sensing principle that detects many of the interferents of concern by probing catalyst poisoning directly. Additionally, it utilizes a refillable water reservoir to keep the membrane hydrated. However, the overall design is not particularly practical in a field setting since the reservoir is required to be refilled with deionized water during maintenance to keep the membrane hydrated. Additionally, the electrochemical cell design and operation are limiting in response time and sensitivity.

### SUMMARY OF THE INVENTION

It is an object of the present invention to provide a novel hydrogen gas sensor.

It is another object of the present invention to provide a hydrogen gas sensor as described above that addresses at least some of the shortcomings associated with existing hydrogen gas sensors.

Therefore, according to one aspect of the invention, there is provided a hydrogen gas sensor, the hydrogen gas sensor comprising: (a) a housing, the housing including a cavity and an aperture, the aperture permitting gas from outside the housing to enter the cavity; (b) a first proton exchange membrane, the first proton exchange membrane being disposed within the cavity; (c) a working electrode, the working electrode being disposed within the cavity and coupled to the first proton exchange membrane; (d) a reference electrode, the reference electrode being disposed within the cavity and coupled to the first proton exchange membrane; and (e) a first counter electrode, the first counter electrode being disposed within the cavity and coupled to the first proton exchange membrane, wherein the first counter electrode comprises one or more materials with pseudo-capacitor characteristics capable of proton intercalation.

In a more detailed feature of the invention, the one or more materials with pseudo-capacitor characteristics capable of proton intercalation may be at least one member selected from the group consisting of transition metal oxides, transition metal sulfides, and electron-conducting polymers.

In a more detailed feature of the invention, the one or more materials with pseudo-capacitor characteristics capable of proton intercalation may be at least one member selected from the group consisting of ruthenium oxide, tungsten oxide, titanium oxide, vanadium oxide, iridium oxide, iron oxide, manganese oxide, and titanium sulfide.

In a more detailed feature of the invention, the one or more materials with pseudo-capacitor characteristics capable of proton intercalation may comprise ruthenium oxide.

In a more detailed feature of the invention, the working electrode may have a working electrode surface area, the first counter electrode may have a first counter electrode surface area, and the first counter electrode surface area may be greater than the working electrode surface area.

In a more detailed feature of the invention, the first counter electrode surface area may be at least about twice the working electrode surface area.

In a more detailed feature of the invention, the hydrogen gas sensor may further comprise a second counter electrode, and the second counter electrode may be disposed within the cavity and coupled to the first proton exchange membrane.

In a more detailed feature of the invention, the second counter electrode may have a second counter electrode surface area, and the second counter electrode surface area may be greater than the first counter electrode surface area.

In a more detailed feature of the invention, the working electrode may have a working electrode surface area, the reference electrode may have a reference electrode surface area, the first counter electrode may have a first counter electrode surface area, the second counter electrode may have a second counter electrode surface area, the reference electrode surface area may be substantially equal to the working electrode surface area, the first counter electrode surface area may be at least about twice as great as each of the working electrode surface area and the reference electrode surface area individually, and the second counter electrode surface area may be greater than the first counter electrode surface area.

In a more detailed feature of the invention, each of the working electrode and the second counter electrode may comprise one or more noble metal electrocatalyst materials.

In a more detailed feature of the invention, the one or more noble metal electrocatalyst materials may be at least one member selected from the group consisting of platinum, palladium, gold, and alloys thereof.

In a more detailed feature of the invention, the reference electrode may comprise one or more pseudo-reference electrode materials.

In a more detailed feature of the invention, the one or more pseudo-reference electrode materials may be at least one member selected from the group consisting of silver, a silver halide, gold, platinum, and platinum black.

In a more detailed feature of the invention, the hydrogen gas substrate may further comprise a substrate, the substrate may comprise opposing top and bottom surfaces, each of the working electrode, the reference electrode, and the first counter electrode may be disposed over the top surface of the substrate, and at least a portion of the first proton exchange membrane may be disposed over and in direct contact with each of the working electrode, the reference electrode, and the first counter electrode.

In a more detailed feature of the invention, the substrate may be made of one or more electrically non-conductive, chemically inert materials.

In a more detailed feature of the invention, the hydrogen gas sensor may further comprise a second counter electrode, the second counter electrode may be disposed over the top surface of the substrate, and at least a portion of the first proton exchange membrane may be disposed over and in direct contact with the second counter electrode.

In a more detailed feature of the invention, the hydrogen gas sensor may further comprise a first contact pad, a second contact pad, a third contact pad, and a fourth contact pad, the first contact pad may be disposed on the substrate outside the cavity and may be electrically coupled to the working electrode by a first trace, the second contact pad may be disposed on the substrate outside the cavity and may be electrically coupled to the reference electrode by a second trace, the third contact pad may be disposed on the substrate outside the cavity and may be electrically coupled to the first counter electrode by a third trace, and the fourth contact pad may be disposed on the substrate outside the cavity and may be electrically coupled to the second counter electrode by a fourth trace.

In a more detailed feature of the invention, the hydrogen gas sensor may further comprise a dielectric film, and the dielectric film may be positioned over at least a portion of each of the first trace, the second trace, the third trace, and the fourth trace.

In a more detailed feature of the invention, the hydrogen gas sensor may further comprise a permselective coating, and the permselective coating may be disposed on the first proton exchange membrane to inhibit interfering gas species from reaching one or more of the working electrode, the reference electrode, and the first counter electrode.

In a more detailed feature of the invention, the permselective coating may have a thickness of about 100 to 1000 microns and may comprise at least one material selected from the group consisting of polymethylmethacrylate, fluorinated ethylene propylene, polyaniline, polytetrafluoroethylene (PTFE), and polyvinylidene fluoride (PVDF).

In a more detailed feature of the invention, the first proton exchange membrane may comprise a perfluorosulfonic acid polymer.

In a more detailed feature of the invention, the first proton exchange membrane may have a thickness of about 50 to 500 microns.

In a more detailed feature of the invention, the hydrogen gas sensor may further comprise a sorbent material containing water for use in keeping the first proton exchange membrane hydrated, and the sorbent material may be disposed within the cavity and coupled to the first proton exchange membrane.

In a more detailed feature of the invention, the hydrogen gas sensor may further comprise a protective barrier, and the protective barrier may be positioned in the cavity to block particulate matter and water from reaching at least one of the working electrode, the reference electrode, and the first counter electrode.

In a more detailed feature of the invention, the protective barrier may comprise at least one gas permeable material selected from the group consisting of a porous polytetrafluoroethylene (PTFE), carbon paper, carbon fiber paper, and silicone.

In a more detailed feature of the invention, the first proton exchange membrane may have opposing first and second surfaces, the working electrode may have opposing first and second surfaces, the first surface of the working electrode may be positioned in direct contact with the first surface of the first proton exchange membrane, and the first surface of the first counter electrode may be positioned in direct contact with the second surface of the first proton exchange membrane.

In a more detailed feature of the invention, the reference electrode may have opposing first and second surfaces, and the first surface of the reference electrode may be positioned in direct contact with the first surface of the first proton exchange membrane.

In a more detailed feature of the invention, the hydrogen gas sensor may further comprise a second proton exchange membrane, the second proton exchange membrane may be disposed within the cavity, the second proton exchange membrane may have opposing first and second surfaces, and the second surface of the first counter electrode may be in direct contact with the first surface of the second polymer exchange membrane.

In a more detailed feature of the invention, the hydrogen gas sensor may further comprise a second counter electrode, the second counter electrode may be disposed within the cavity, the second counter electrode may have opposing first and second surfaces, and the first surface of the second counter electrode may be positioned in direct contact with the second surface of the second proton exchange membrane.

In a more detailed feature of the invention, the hydrogen gas sensor may further comprise a first current collector, a second current collector, a third current collector, and a fourth current collector, the first current collector may be positioned between the first proton exchange membrane and the second proton exchange membrane and may be electrically coupled to the first counter electrode, the second current collector may be positioned along the second surface of the second proton exchange membrane and may be electrically coupled to the second counter electrode, the third current collector may be positioned along the first surface of the first proton exchange membrane and may be electrically coupled to the working electrode, and the fourth current collector may be positioned along the first proton exchange membrane and may be electrically coupled to the reference electrode.

In a more detailed feature of the invention, the hydrogen gas sensor may further comprise a first protective barrier and a second protective barrier, the first protective barrier may be positioned outside the third and fourth current collectors to block particulate matter and water from reaching the working electrode and the reference electrode, and the second protective barrier may be positioned outside the second current collector to block particulate matter and water from reaching the second counter electrode.

According to another aspect of the invention, there is provided a method for assessing hydrogen gas purity, the method comprising the steps of: (a) providing a hydrogen gas sensor, the hydrogen gas sensor comprising (i) a proton exchange membrane, (ii) a working electrode, the working electrode coupled to the proton exchange membrane, (iii) a reference electrode, the reference electrode coupled to the proton exchange membrane, and (iv) a first counter electrode, the first counter electrode coupled to the proton exchange membrane and comprising one or more materials with pseudo-capacitor characteristics capable of proton intercalation; (b) applying a first potential difference between the working electrode and the reference electrode; (c) exposing a hydrogen gas sample to the working electrode, whereby hydrogen gas is oxidized at the working electrode and protons travel from the working electrode to the first counter electrode via the proton exchange membrane and are stored in the first counter electrode; (d) measuring an oxidation current as the hydrogen gas sample is oxidized; and (e) comparing the measured oxidation current to standards to assess hydrogen gas purity.

In a more detailed feature of the invention, the method may further comprise, after step (d), applying a second potential difference between the working electrode and the reference electrode to strip any contaminants from the working electrode.

In a more detailed feature of the invention, the method may further comprise comparing the second potential difference used to strip the contaminants to standards to identify the contaminants.

In a more detailed feature of the invention, the one or more materials with pseudo-capacitor characteristics capable of proton intercalation may be at least one member selected from the group consisting of transition metal oxides, transition metal sulfides, and electron-conducting polymers.

In a more detailed feature of the invention, the one or more materials with pseudo-capacitor characteristics capable of proton intercalation may be at least one member selected from the group consisting of ruthenium oxide, tungsten oxide, titanium oxide, vanadium oxide, iridium oxide, iron oxide, manganese oxide, and titanium sulfide.

In a more detailed feature of the invention, the one or more materials with pseudo-capacitor characteristics capable of proton intercalation may comprise ruthenium oxide.

In a more detailed feature of the invention, the working electrode may have a working electrode surface area, the first counter electrode may have a first counter electrode surface area, and the first counter electrode surface area may be greater than the working electrode surface area.

In a more detailed feature of the invention, the first counter electrode surface area may be at least about twice the working electrode surface area.

According to yet another aspect of the invention, there is provided a method for quantitating hydrogen gas, the method comprising the steps of (a) providing a hydrogen gas sensor, the hydrogen gas sensor comprising (i) a proton exchange membrane, (ii) a working electrode, the working electrode coupled to the proton exchange membrane, (iii) a reference electrode, the reference electrode coupled to the proton exchange membrane, (iv) a first counter electrode, the first counter electrode coupled to the proton exchange membrane and comprising one or more materials with pseudo-capacitor characteristics capable of proton intercalation; (v) a second counter electrode, the second counter electrode coupled to the proton exchange membrane; (b) applying a potential difference between the working electrode and the reference electrode; (c) exposing a sample to the working electrode, whereby hydrogen gas, if present, is oxidized at the working electrode to generate protons that travel from the working electrode to the first counter electrode via the proton exchange membrane and are intercalated in the first counter electrode; (d) measuring an oxidation current for the sample; (e) comparing the measured oxidation current to standards to quantitate hydrogen gas.

In a more detailed feature of the invention, the method may further comprise, after step (d), the steps of applying a potential difference between the first counter electrode and the reference electrode to cause protons intercalated in the first counter electrode to be de-intercalated therefrom and to travel, via the proton exchange membrane, to the second counter electrode; measuring a discharge current profile for the protons de-intercalated from the first counter electrode; and comparing the discharge current profile to standards to quantitate hydrogen gas.

In a more detailed feature of the invention, the one or more materials with pseudo-capacitor characteristics capable of proton intercalation may be at least one member selected from the group consisting of transition metal oxides, transition metal sulfides, and electron-conducting polymers.

In a more detailed feature of the invention, the one or more materials with pseudo-capacitor characteristics capable of proton intercalation may be at least one member selected from the group consisting of ruthenium oxide, tungsten oxide, titanium oxide, vanadium oxide, iridium oxide, iron oxide, manganese oxide, and titanium sulfide.

In a more detailed feature of the invention, the one or more materials with pseudo-capacitor characteristics capable of proton intercalation may comprise ruthenium oxide.

In a more detailed feature of the invention, the working electrode may have a working electrode surface area, the first counter electrode may have a first counter electrode surface area, and the first counter electrode surface area may be greater than the working electrode surface area.

In a more detailed feature of the invention, the first counter electrode surface area may be at least about twice the working electrode surface area.

In a more detailed feature of the invention, the first counter electrode may have a first counter electrode surface area, the second counter electrode may have a second counter electrode surface area, and the second counter electrode surface area may be greater than the first counter electrode surface area.

According to still yet another aspect of the invention, there is provided a method for quantitating hydrogen gas, the method comprising the steps of (a) providing a hydrogen gas sensor, the hydrogen gas sensor comprising (i) a proton exchange membrane, (ii) a working electrode, the working electrode coupled to the proton exchange membrane, (iii) a reference electrode, the reference electrode coupled to the proton exchange membrane, (iv) a first counter electrode, the first counter electrode coupled to the proton exchange membrane and comprising one or more materials with pseudo-capacitor characteristics capable of proton intercalation; (v) a second counter electrode, the second counter electrode coupled to the proton exchange membrane; (b) applying a potential difference between the working electrode and the reference electrode; (c) exposing a sample to the working electrode for a measured period of time, whereby hydrogen gas, if present, is oxidized at the working electrode to generate protons that travel from the working electrode to the first counter electrode via the proton exchange membrane and are intercalated in the first counter electrode; (d) applying a potential difference between the first counter electrode and the reference electrode to cause protons intercalated in the first counter electrode to be de-intercalated therefrom and to travel, via the proton exchange membrane, to the second counter electrode; (e) measuring a discharge current profile for the protons de-intercalated from the first counter electrode; and (f) comparing the discharge current profile to standards to quantitate hydrogen gas.

In a more detailed feature of the invention, the one or more materials with pseudo-capacitor characteristics capable of proton intercalation may be at least one member selected from the group consisting of transition metal oxides, transition metal sulfides, and electron-conducting polymers.

In a more detailed feature of the invention, the one or more materials with pseudo-capacitor characteristics capable of proton intercalation may be at least one member selected from the group consisting of ruthenium oxide, tungsten oxide, titanium oxide, vanadium oxide, iridium oxide, iron oxide, manganese oxide, and titanium sulfide.

In a more detailed feature of the invention, the one or more materials with pseudo-capacitor characteristics capable of proton intercalation may comprise ruthenium oxide.

In a more detailed feature of the invention, the working electrode may have a working electrode surface area, the first counter electrode may have a first counter electrode surface area, and the first counter electrode surface area may be greater than the working electrode surface area.

In a more detailed feature of the invention, the first counter electrode surface area may be at least about twice the working electrode surface area.

In a more detailed feature of the invention, the first counter electrode may have a first counter electrode surface area, the second counter electrode may have a second counter electrode surface area, and the second counter electrode surface area may be greater than the first counter electrode surface area.

The present invention is also directed at systems using the above-described hydrogen gas sensor for quantitating hydrogen gas and/or for assessing hydrogen gas purity.

The present invention is further directed at methods for making the above-described hydrogen gas sensor.

Additional objects, as well as aspects, features, and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. The drawings are not necessarily drawing to scale, and certain components may have undersized and/or oversized dimensions for purposes of explication. In the drawings wherein like reference numeral represents like parts:

FIG. 1 is a top view of a first embodiment of a hydrogen gas sensor constructed according to the present invention;

FIG. 2 is an enlarged section view of the hydrogen gas sensor of FIG. 1 taken along line 2-2;

FIG. 3 is a top view of the hydrogen gas sensor of FIG. 1, with certain components not being shown to reveal other components that would otherwise be hidden;

FIG. 4 is a flowchart depicting one embodiment of a method according to the present invention by which the hydrogen gas sensor of FIG. 1 may be used to assess the purity of a hydrogen gas sample;

FIG. 5 is a flowchart depicting a first embodiment of a method according to the present invention by which the hydrogen gas sensor of FIG. 1 may be used to quantitate hydrogen gas;

FIG. 6 is a flowchart depicting a second embodiment of a method according to the present invention by which the hydrogen gas sensor of FIG. 1 may be used to quantitate hydrogen gas;

FIG. 7 is a diagram schematically depicting a mechanism by which the hydrogen gas sensor of FIG. 1 may be used according to the present invention for hydrogen gas quantitation;

FIG. 8 is a graph illustrating how, for the hydrogen gas sensor of FIG. 1, an oxidation current may be used to measure hydrogen gas at higher levels whereas a discharge profile may be used to detect hydrogen gas at lower levels;

FIG. 9 is a graph illustrating how, for the hydrogen gas sensor of FIG. 1, a drop in current may be used to assess hydrogen gas purity;

FIG. 10 is a simplified schematic view of one embodiment of a system that includes the hydrogen gas sensor of FIG. 1;

FIG. 11 is a top view of a second embodiment of a hydrogen gas sensor constructed according to the present invention;

FIG. 12 is an enlarged section view of the hydrogen gas sensor of FIG. 11 taken along line 12-12;

FIG. 13 is a top view of a third embodiment of a hydrogen gas sensor constructed according to the present invention;

FIG. 14 is an enlarged section view of the hydrogen gas sensor of FIG. 13 taken along line 14-14;

FIG. 15 is a partly exploded perspective view of the hydrogen gas sensor of FIG. 13; and

FIG. 16 is a simplified schematic view of one embodiment of a system that includes the hydrogen gas sensor of FIG. 13.

### DETAILED DESCRIPTION OF THE INVENTION

The present invention is based, in part, on the surprising discovery of a novel hydrogen gas sensor, as well as systems including the aforementioned hydrogen gas sensor, and is based, in part, on the surprising discovery of various methods of using the aforementioned hydrogen gas sensor to quantitate hydrogen gas and/or to assess hydrogen gas purity.

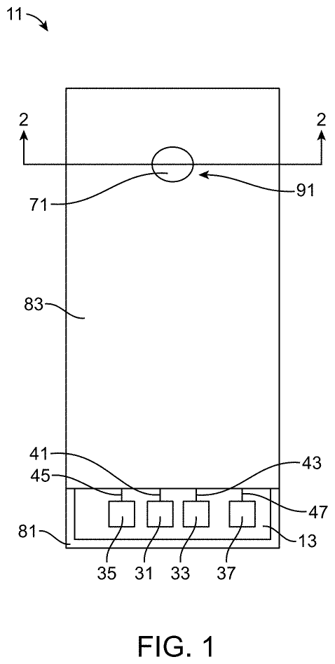

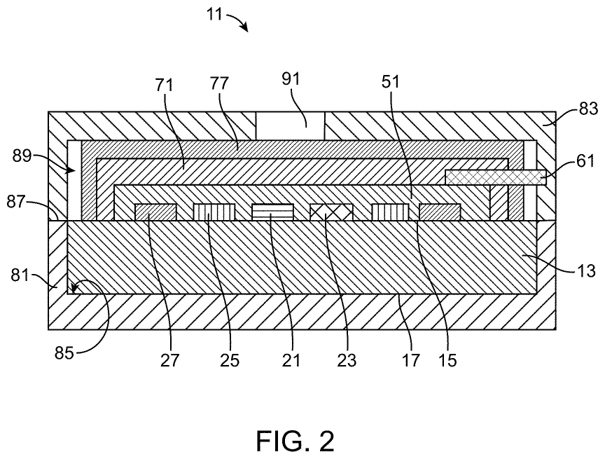

Referring now to FIGS. 1 through 3, there are shown various views of a first embodiment of a hydrogen gas sensor constructed according to the present invention, the hydrogen gas sensor being represented generally by reference numeral 11. Details of hydrogen gas sensor 11 that are discussed elsewhere in this application or that are not critical to an understanding of the invention may be omitted from one or more of FIGS. 1, 2, and 3 and/or from the accompanying description herein or may be shown in one or more of FIGS. 1, 2, and 3 and/or described herein in a simplified manner.

Hydrogen gas sensor 11 may comprise a substrate 13. Substrate 13 may be a generally rectangular, planar structure made of a rigid, electrically non-conductive, chemically inert material, such as a suitable plastic or ceramic. Substrate 13 may comprise a top surface 15 and a bottom surface 17.

Hydrogen gas sensor 11 may further comprise a plurality of electrodes, wherein said plurality of electrodes may be spaced apart from one another. In the present embodiment, the plurality of electrodes may comprise a working electrode 21, a reference electrode 23, a first counter electrode 25, and a second counter electrode 27. Notwithstanding the above, as will be discussed further below, for certain applications, hydrogen gas sensor 11 need not include a second counter electrode; thus, in another embodiment, second counter electrode 27 may be omitted.

Working electrode 21, which may be positioned directly on top of top surface 15 of substrate 13, may consist of or comprise one or more noble metal electrocatalyst materials, said one or more noble metal electrocatalyst materials being at least one member selected from the group including, but not being limited to, platinum, palladium, gold, and alloys thereof. As will be discussed further below, working electrode 21 may be used to oxidize hydrogen gas in a sample, thereby producing protons.

Reference electrode 23, which may also be positioned directly on top of top surface 15 of substrate 13, may consist of or comprise one or more suitable pseudo-reference electrode materials, said one or more such materials being at least one member selected from the group including, but not being limited to, silver, a silver halide (e.g., chloridized silver), gold, platinum, and platinum black.

First counter electrode 25, which may also be positioned directly on top of top surface 15 of substrate 13, may consist of or comprise one or more pseudocapacitance materials, i.e., materials with pseudo-capacitor characteristics capable of proton intercalation. Pseudocapacitance is a phenomenon that describes the electrochemical charge storage that occurs at the surface of a material due to reversible faradaic redox reactions. In contrast to the electrostatic charge storage in traditional capacitors, which relies on the separation of charge at the interface between two conductive materials, pseudocapacitance is based on surface redox reactions that involve the transfer of electrons between the electrode surface and the electrolyte. The term “pseudo” is used because the charge storage mechanism is not purely electrostatic like in traditional capacitors, but rather involves redox reactions that are more similar to those that occur in batteries. However, unlike batteries, pseudocapacitors can typically deliver high power densities and exhibit faster charge/discharge rates, making them attractive for applications that require rapid energy storage and release. Pseudocapacitance can occur in a variety of materials, including metal oxides, metal sulfides, and electron-conducting polymers, as well as other electrode materials that have active redox sites at their surface. The magnitude of the pseudocapacitance is determined by factors such as the surface area of the electrode, the nature of the redox-active species, and the conductivity of the electrode material. In view of the above, materials that may be suitable for use as first counter electrode 25 may include metal oxides, metal sulfides, and electron-conducting polymers of the type described above. Examples of suitable metal oxides and metal sulfides include, but are not limited to, one or more of the following: ruthenium oxide (RuO₂), tungsten oxide (W₂O₃), titanium oxide (TiO₂), vanadium oxide (V₂O₅), iridium oxide (IrO₂), iron oxide (Fe₃O₄), manganese oxide (MnO₂), and titanium sulfide (TiS₂). Of these materials, ruthenium oxide may be particularly desirable. As will be discussed further below, first counter electrode 25 may be used to store, typically temporarily, protons generated by the oxidation of hydrogen gas at working electrode 21.

Second counter electrode 27, which may also be positioned directly on top of top surface 15 of substrate 13, may consist of or comprise one or more noble metal electrocatalyst materials, said one or more noble metal electrocatalyst materials being at least one member selected from the group including, but not being limited to, platinum, palladium, gold, and alloys thereof. As will be discussed further below, second counter electrode 27 may be used to receive protons that may be discharged from first counter electrode 25 and to reduce such protons.

Working electrode 21, reference electrode 23, first counter electrode 25, and second counter electrode 27 may be fabricated by similar or different processes. Processes that may be used to form one or more of working electrode 21, reference electrode 23, first counter electrode 25, and second counter electrode 27 may include, but are not limited to, sputtering, spray coating, screen printing, metal deposition, etc. In particular, where first counter electrode 25 is made of a metal oxide or a metal sulfide, electrodeposition processes may be used to form first counter electrode 25.

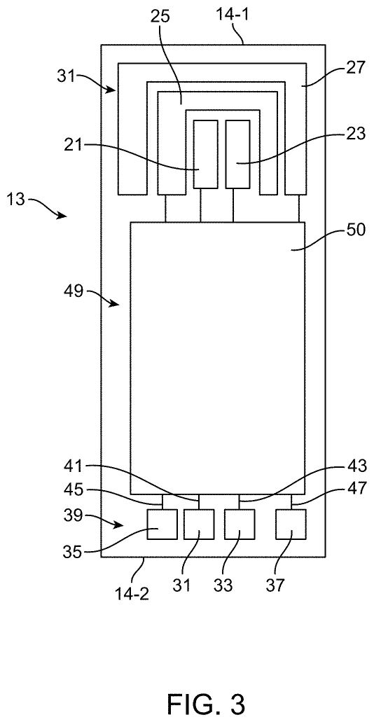

As can be seen best in FIG. 3, working electrode 21, reference electrode 23, first counter electrode 25, and second counter electrode 27 may collectively form a sensing region 31 proximate to a first end 14-1 of substrate 13. Working electrode 21 and reference electrode 23, which may be positioned generally parallel to one another, each may have a generally rectangular surface area or footprint, with the surface areas of working electrode 21 and reference electrode 23 being comparable to one another. First counter electrode 25, which may have a generally C-shaped footprint and which may surround the combination of working electrode 21 and reference electrode 23 on three sides, may have a surface area (and corresponding volume) that is considerably greater (e.g., nearly double or more) than that of working electrode 21. Second counter electrode 27, which may have a generally C-shaped footprint and which may surround first counter electrode 25 on three sides, may have a surface area (and corresponding volume) that is considerably greater than that of first counter electrode 25. It is to be understood that, although working electrode 21, reference electrode 23, first counter electrode 25, and second counter electrode 27 have the shapes discussed above, the present invention is not limited to such shapes; consequently, working electrode 21, reference electrode 23, first counter electrode 25, and second counter electrode 27 could be sized, shaped or positioned differently, and, as a result, other components of hydrogen gas sensor 11 could be adjusted accordingly.

Hydrogen gas sensor 11 may further comprise a plurality of contact pads, wherein said contact pads may be spaced apart from one another. In the present embodiment, the plurality of contact pads may comprise a first contact pad 31, a second contact pad 33, a third contact pad 35, and a fourth contact pad 37. Each of first contact pad 31, second contact pad 33, third contact pad 35 and fourth contact pad 37 may be positioned directly on top of top surface 15 of substrate 13 proximate to a second end 14-2 of substrate 13, thereby forming a contact pad region 39 on substrate 13. First contact pad 31, second contact pad 33, third contact pad 35, and fourth contact pad 37 may be similar to one another in size, shape, and composition and may be formed by a similar process. In the present embodiment, each of first contact pad 31, second contact pad 33, third contact pad 35, and fourth contact pad 37 may consist of or comprise an electrically-conductive material, such as a suitable metal (e.g., gold, silver, etc.), and may be fabricated by a process that may include, but is not limited to, sputtering, spray coating, screen printing, metal deposition, etc.

Hydrogen gas sensor 11 may further comprise a plurality of traces or leads. In the present embodiment, the plurality of traces or leads may comprise a first trace 41, a second trace 43, a third trace 45, and a fourth trace 47. Each of first trace 41, second trace 43, third trace 45 and fourth trace 47 may be positioned directly on top of top surface 15 of substrate 13 between sensing region 31 and contact pad region 39, thereby forming a trace region 49 on substrate 13.

First trace 41 may be coupled at a first end to working electrode 21 and may be coupled at a second end to first contact pad 31. Second trace 43 may be coupled at a first end to reference electrode 23 and may be coupled at a second end to second contact pad 33. Third trace 45 may be coupled at a first end to first counter electrode 25 and may be coupled at a second end to third contact pad 35. Fourth trace 47 may be coupled at a first end to second counter electrode 27 and may be coupled at a second end to fourth contact pad 37. First trace 41, second trace 43, third trace 45, and fourth trace 47 may be similar to one another in size, shape, and composition and may be formed by a similar process. In the present embodiment, each of first trace 41, second trace 43, third trace 45, and fourth trace 47 may consist of or comprise an electrically-conductive material, such as a suitable metal (e.g., gold, silver, etc.), and may be fabricated by a process that may include, but is not limited to, sputtering, spray coating, screen printing, metal deposition, etc.

As can be appreciated, where the hydrogen gas sensor omits second counter electrode 27, fourth contact pad 37 and fourth trace 47 may also be omitted.

Hydrogen gas sensor 11 may further comprise a dielectric film 50, which may be used to electrically insulate most of trace region 49. In the present embodiment, dielectric film 50 may be appropriately dimensioned to cover most of trace region 49. More specifically, dielectric film 50 may be positioned directly over most of first trace 41, second trace 43, third trace 45, and fourth trace 47.

Hydrogen gas sensor 11 may further comprise a proton exchange membrane 51. In the present embodiment, proton exchange membrane 51 is preferably a non-porous, proton-conductive, electrically-non-conductive, liquid permeable and gas permeable membrane. Proton exchange membrane 51 may consist of or comprise a homogeneous perfluorosulfonic acid (PFSA) polymer. Said PFSA polymer may be formed by the copolymerization of tetrafluoroethylene and perfluorovinylether sulfonic acid. See e.g., U.S. Pat. No. 3,282,875, inventors Connolly et al., issued Nov. 1, 1966; U.S. Pat. No. 4,470,889, inventors Ezzell et al., issued Sep. 11, 1984; U.S. Pat. No. 4,478,695, inventors Ezzell et al., issued Oct. 23, 1984; and U.S. Pat. No. 6,492,431, inventor Cisar, issued Dec. 10, 2002, all of which are incorporated herein by reference in their entireties. Examples of materials that may be suitable for use as proton exchange membrane 51 may include the following PFSA polymer membranes commercialized by The Chemours Company FC, LLC (Wilmington, Del.) as NAFION™ extrusion cast PFSA polymer membranes: NAFION™ 115 PFSA polymer membrane, NAFION™ XL PFSA polymer membrane, NAFION™ 117 PFSA polymer membrane, and NAFION™ 1100W PFSA polymer membrane. Additional examples of materials that may be suitable for use as proton exchange membrane 51 may include the following cation exchange membranes commercialized by Fumatech BWT GmbH (Bietigheim-Bissingen, Germany): FUMASEP FKS-30 cation exchange membrane, FUMASEP FKS-50 cation exchange membrane, FUMASEP F-1850 cation exchange membrane, FUMAPEM F-950 cation exchange membrane, and FUMAPEM F-14100 cation exchange membrane. Still other examples of materials that may be suitable for use as proton exchange membrane 51 may include the following cation exchange membranes commercialized by Solvay Specialty Polymers USA, LLC (Greenville, S.C.): AQUIVION® E98-095 PFSA polymer membrane and AQUIVION® E98-15S PFSA polymer membrane.

Proton exchange membrane 51, which may have a thickness of about 50 μm to 500 μm, may be in the form of a unitary (i.e., one-piece) structure and may be appropriately dimensioned to cover the entirety of sensing region 31, as well as a surrounding area of substrate 13. More specifically, proton exchange membrane 51 may be in direct contact with the top surface of each of working electrode 21, reference electrode 23, first counter electrode 25, and second counter electrode 27, as well as top surface 15 of substrate 13. Proton exchange membrane 51 may be formed over sensing region 31 and a surrounding area of substrate 15, for example, by dip-coating or spray-coating the electrode-covered substrate with a suspension comprising the proton exchange membrane material in a suitable organic solvent.

Hydrogen gas sensor 11 may further comprise a sorbent material 61. Sorbent material 61, which may be saturated with water, may be used to help keep proton exchange membrane 51 hydrated. In the present embodiment, sorbent material 61 may be placed in direct contact with proton exchange membrane 51, for example, by being positioned directly on top of proton exchange membrane 51 at or along its periphery.

Hydrogen gas sensor 11 may further comprise a permselective coating 71. Permselective coating 71 may serve to inhibit the diffusion of interfering gas species from reaching sensing region 31. In the present embodiment, permselective coating 71 may consist of or comprise one or more materials selected from the group including, but not limited to, polymethylmethacrylate, fluorinated ethylene propylene, polyaniline, polytetrafluoroethylene (PTFE), polyvinylidene fluoride (PVDF), and other related polymers. Permselective coating 71 may be dimensioned and positioned so that it covers the entirety of proton exchange membrane 51, except for that portion of proton exchange membrane 51 that is contacted by sorbent material 61, and also may cover a surrounding area of substrate 13. Permselective coating 71 may have a thickness of about 100 μm to 1000 μm and may be formed by dip coating, spray coating, or spin coating.

Hydrogen gas sensor 11 may further comprise a protective barrier 77. Protective barrier 77 may serve to block particulate matter and water from reaching sensing region 31. In the present embodiment, protective barrier 77 may consist of or comprise one or more materials selected from the group including, but not limited to a porous polytetrafluoroethylene (PTFE), carbon paper, carbon fiber paper, silicone, or other gas permeable materials. Protective barrier 77 may be dimensioned, for example, by die cutting, to a size that covers sensing region 31, as well as a surrounding area of substrate 13, and may be placed directly on top of permselective coating 71.

Hydrogen gas sensor 11 may further comprise a sensor housing. In the present embodiment, the sensor housing may comprise a bottom portion 81 and a top portion 83. Bottom portion 81 may be a unitary (i.e., one-piece) structure, which may be made by injection molding and which may consist of or comprise a suitable plastic material, such as, but not limited to, an acrylic, an acrylonitrile butadiene styrene, a nylon, a polycarbonate, a polyethylene, a polypropylene, a polystyrene, etc. In the present embodiment, bottom portion 81 may be shaped to include a cavity 85. Cavity 85 may be appropriately dimensioned to receive substrate 13, with top surface 15 of substrate 13 lying substantially flush with a top edge 87 of bottom portion 81.

Top portion 83 may be a unitary (i.e., one-piece) structure, which may be made by injection molding and which may consist of or comprise a suitable plastic material, such as, but not limited to, an acrylic, an acrylonitrile butadiene styrene, a nylon, a polycarbonate, a polyethylene, a polypropylene, a polystyrene, etc. Top portion 83, which may be removably or permanently joined to bottom portion 81, may be shaped to include a cavity 89. In the present embodiment, cavity 89 may be appropriately dimensioned to receive sensing region 31, proton exchange membrane 51, sorbent material 61, permselective coating 71, and protective barrier 77, as well as dielectric film 50 and most of trace region 41, thereby leaving only contact pad region 39 and a small portion of each of traces 41, 43, 45, and 47 exposed.

Top portion 83 of the sensor housing may also be shaped to include an aperture or opening 91. In the present embodiment, aperture 91 may be positioned over sensing region 31 to allow gas to enter the sensor housing for analysis.

In addition, although not shown, hydrogen gas sensor 11 may additionally comprise one or more gaskets or other hardware that may be used, for example, to seal the electronic components from moisture and/or to prevent moisture from escaping sorbent material 61. Such hardware may seal the sensor so that only a small aperture exposes the sensor and is in direct contact with the protective barrier layer. The hardware also may seal the sorbent material as to ensure membrane hydration and prevent moisture from reaching the board electronics. Said hardware may also be designed in a form factor where it can be easily mounted to surfaces.

Also, although not shown, hydrogen gas sensor 11 may include a second working electrode that may be used solely to calibrate working electrode 21. Alternatively, said second working electrode may be omitted, and working electrode 21 may be factory calibrated.

The hydrogen gas sensor of the present invention may be capable of being used in a plurality of alternative modes of operation. For example, according to one embodiment, the hydrogen gas sensor of the present invention may be used to assess hydrogen gas purity. As another example, according to another embodiment, the hydrogen gas sensor of the present invention may be used to quantitate hydrogen gas. Such quantitation may be used to detect hydrogen gas levels at moderately low concentration levels (e.g., parts per million up to a few %) or at very low concentration levels (e.g., parts per billion). Details of these various alternative modes of operation are discussed below. As will be seen, all of these modes of operation utilize the pseudocapacitive characteristics of the first counter electrode to reversibly store protons that have been generated by the oxidation of hydrogen gas.

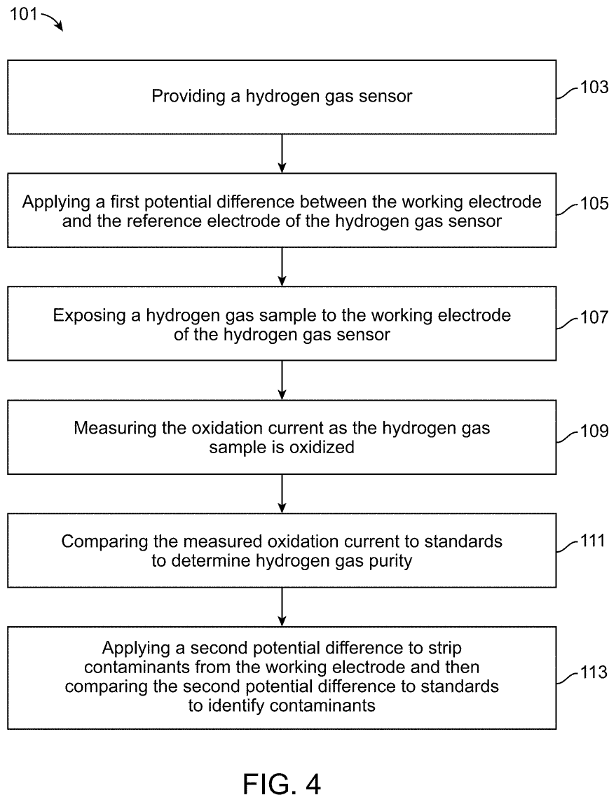

Referring now to FIG. 4, there is shown a flowchart illustrating one embodiment of a method according to the present invention by which a hydrogen gas sensor may be used to assess the purity of a hydrogen gas sample, the method being represented generally by reference numeral 101.

Method 101 may comprise a step 103 of providing a hydrogen gas sensor. The hydrogen gas sensor of step 103 may comprise a proton exchange membrane to which a working electrode, a reference electrode, and a first counter electrode are coupled. Each of the proton exchange membrane, the working electrode, the reference electrode, and the first counter electrode may have a composition that is identical to the corresponding component of hydrogen gas sensor 11. Consequently, the hydrogen gas sensor of step 103 may be similar or identical to hydrogen gas sensor 11. Notwithstanding the above, the hydrogen gas sensor of step 103 may omit a second counter electrode (like second counter electrode 27 of hydrogen gas sensor 11). Also, although the hydrogen gas sensor of step 103 preferably has a first counter electrode whose surface area is greater than that of its working electrode, the surface area of the first counter electrode does not necessarily need to be greater than that of the working electrode.

Method 101 may further comprise a step 105 of applying a voltage between the working electrode and the reference electrode of the hydrogen gas sensor.

Method 101 may further comprise a step 107 of exposing a hydrogen gas sample to the working electrode of the hydrogen gas sensor. As the hydrogen gas sample arrives at the working electrode, the hydrogen gas becomes oxidized, thereby forming protons. The thus-formed protons travel through the proton exchange membrane to the first counter electrode, where the protons become stored or intercalated within the first counter electrode.

Method 101 may further comprise a step 109 of measuring the oxidation current as the hydrogen gas sample is oxidized. If contaminants are present in the hydrogen gas sample, there will be a decrease in the oxidation current. By contrast, if no contaminants are present in the hydrogen gas sample, the oxidation current will stay constant (at least until the first counter electrode is incapable of storing any additional protons).

Method 101 may further comprise a step 111 of comparing the measured oxidation current to standards to permit an assessment of the purity of the sample.

Method 101 may further comprise a step 113 of applying a voltage between the reference electrode and the first counter electrode to strip any contaminants from the working electrode. The voltage that is needed to strip the contaminants may then be compared to standards to identify the composition of the contaminant.

One advantage to using the hydrogen gas sensor of the present invention to assess hydrogen gas purity is that the hydrogen gas sensor of the present invention includes a pseudocapacitance material that stores protons. This is in contrast with conventional hydrogen gas sensors that typically include a counter electrode at which hydrogen gas is evolved. During the operation and periodic stripping processes of these conventional sensors, water is lost from the polymer exchange membrane, causing the polymer exchange membrane to dry out. Because the hydrogen gas sensor of the present invention does not produce hydrogen gas at the cathode, the operation and stripping processes are less apt to remove water from the polymer exchange membrane. As a result, the polymer exchange membrane of the present sensor does not as easily dry out or as frequently require rehydration, and the present sensor can be used for longer durations, all of which are highly desirable.

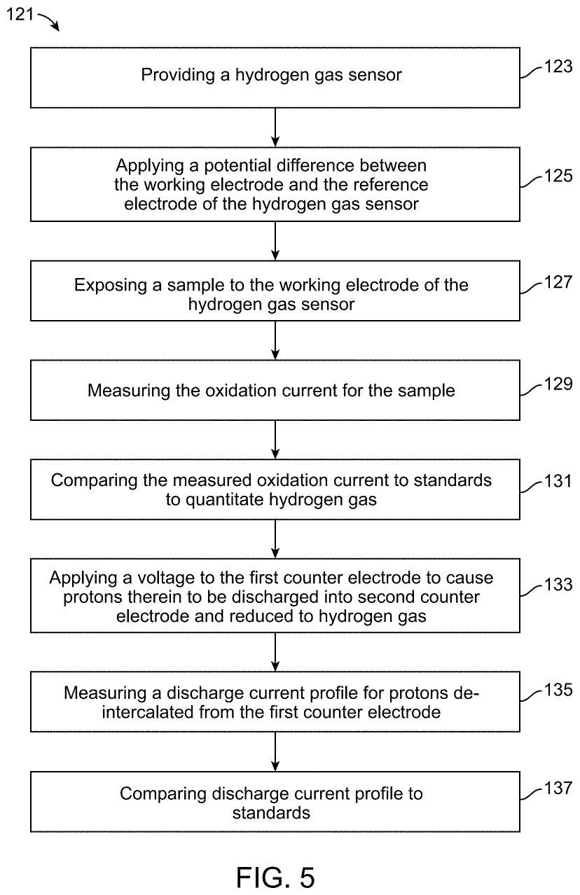

Referring now to FIG. 5, there is shown a flowchart illustrating a first embodiment of a method according to the present invention by which a hydrogen gas sensor may be used to quantitate hydrogen gas, the method being represented generally by reference numeral 121.

Method 121, which is best-suited for detecting hydrogen gas concentrations in the range of ppm to a few %, may comprise a step 123 of providing a hydrogen gas sensor. The hydrogen gas sensor of step 123 may comprise a proton exchange membrane to which a working electrode, a reference electrode, a first counter electrode, and a second counter electrode are coupled. Each of the proton exchange membrane, the working electrode, the reference electrode, the first counter electrode, and the second counter electrode may have a composition that is identical to the corresponding component of hydrogen gas sensor 11. Consequently, the hydrogen gas sensor of step 123 may be similar or identical to hydrogen gas sensor 11. Notwithstanding the above, although the hydrogen gas sensor of step 123 preferably has a second counter electrode whose surface area is greater than that of its first counter electrode, the surface area of the second counter electrode does not necessarily need to be greater than that of the first counter electrode.

Method 121 may further comprise a step 125 of applying a voltage between the working electrode and the reference electrode of the hydrogen gas sensor.

Method 121 may further comprise a step 127 of exposing a sample to the working electrode of the hydrogen gas sensor. (Although step 127 refers to exposing a sample to the working electrode of the hydrogen gas sensor, it is to be understood that step 127 may be performed simply by passively allowing hydrogen gas in a space near the hydrogen gas sensor to contact the working electrode of the hydrogen gas sensor. This may be the case, for example, where method 121 is used to detect a hydrogen gas leak.) If hydrogen gas is present in the sample, as the hydrogen gas reaches the working electrode, the hydrogen gas becomes oxidized, thereby forming protons. The thus-formed protons travel through the proton exchange membrane to the first counter electrode, where the protons become stored or intercalated within the first counter electrode.

Method 121 may further comprise a step 129 of measuring the oxidation current. If no hydrogen gas is present in the sample, no hydrogen gas will be oxidized, and there will be no oxidation current. On the other hand, if hydrogen gas is present, there will be an oxidation current, the current being proportional to the concentration of hydrogen gas that is oxidized. If desired, steps 127 and 129 can be performed until the oxidation current drops significantly, thereby signifying that first counter electrode has become saturated with protons.

Method 121 may further comprise a step 131 of comparing the measured oxidation current to standards to permit a quantitation of hydrogen gas in the sample. This comparison could also take into account the time that elapsed for the first counter electrode to be saturated with protons.

Method 121 may further comprise a step 133 of applying a voltage to the first counter electrode, causing protons stored or intercalated within the first counter electrode to be de-intercalated therefrom and to travel, via the proton exchange membrane, to the second counter electrode, where they may be reduced to form hydrogen gas.

Method 121 may further comprise a step 135 of measuring a discharge current profile for the protons de-intercalated from the first counter electrode.

Method 121 may further comprise a step 137 of comparing the discharge current profile to standards (which may also take into account the time to saturate the first counter electrode with protons if such occurred) to quantitate the amount of hydrogen gas present. The discharge current profile that is measured may, for example, be in the form of a profile or curve, with the area under the profile or curve being proportional to the hydrogen quantification. Additional or alternative characteristics of the discharge current profile may also be used.

It should be noted that the discharge current profile may be used instead of the oxidation current to quantitate the amount of hydrogen gas that is present. Alternatively, the discharge current profile may be compared to the oxidation current to provide confirmation that what was detected using the oxidation current is, in fact, hydrogen gas, as opposed to a contaminant (which remains in the working electrode, as opposed to traveling from the working electrode through the polymer exchange membrane to the first counter electrode). By making the second counter electrode greater in surface area than the first counter electrode, the discharge process can be expedited.

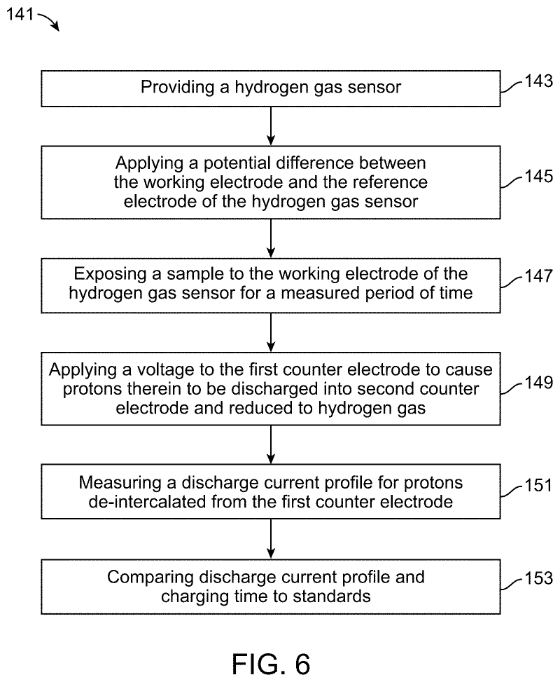

Referring now to FIG. 6, there is shown a flowchart illustrating a second embodiment of a method according to the present invention by which a hydrogen gas sensor may be used to quantitate hydrogen gas, the method being represented generally by reference numeral 141.

Method 141, which is best-suited for detecting hydrogen gas concentrations in the range of ppb, may comprise a step 143 of providing a hydrogen gas sensor. The hydrogen gas sensor of step 143 may be similar or identical to that of method 121. Consequently, the hydrogen gas sensor of step 143 may be similar or identical to hydrogen gas sensor 11.

Method 141 may further comprise a step 145 of applying a voltage between the working electrode and the reference electrode of the hydrogen gas sensor.

Method 141 may further comprise a step 147 of exposing a sample to the working electrode of the hydrogen gas sensor for a measured period of time. (Although step 147 refers to exposing a sample to the working electrode of the hydrogen gas sensor, it is to be understood that step 147 may be performed simply by passively allowing hydrogen gas in a space near the hydrogen gas sensor to contact the working electrode of the hydrogen gas sensor. This may be the case, for example, where method 141 is used to detect a hydrogen gas leak.) If hydrogen gas is present in the sample, as the hydrogen gas reaches the working electrode, the hydrogen gas becomes oxidized, thereby forming protons. The thus-formed protons travel through the proton exchange membrane to the first counter electrode, where the protons become stored or intercalated within the first counter electrode.

Because the concentration of hydrogen gas detected by method 141 is very low (i.e., ppb levels), any oxidation current that is produced is too low to be detectable. Consequently, method 141 does not rely on measuring the oxidation current and comparing the same to standards. Instead, step 147 is performed for a sufficient period of time to allow a significant quantity of protons to accumulate in the first counter electrode. Then, method 141 may further comprise a step 149 of applying a voltage to the first counter electrode, causing protons that are stored or intercalated within the first counter electrode to be de-intercalated therefrom and to travel, via the proton exchange membrane, to the second counter electrode, where they may be reduced to form hydrogen gas. Because the number of protons that are stored or intercalated in the first counter electrode has been allowed to build up over time, the discharge current profile that reflects the de-intercalation of protons from the first counter electrode is likely to be detectable.

Method 141 may further comprise a step 151 of measuring the discharge current profile for the de-intercalated protons.

Method 141 may further comprise a step 153 of comparing the discharge current profile, as well as the time spent charging the first counter electrode with protons, to standards to quantitate the amount of hydrogen gas present. The discharge current profile that is measured may, for example, be in the form of a profile or curve, with the area under the profile or curve being proportional to the hydrogen quantification. Additional or alternative characteristics of the discharge current profile may also be used.

As discussed above, depending on its method of operation, hydrogen gas sensor 11 may be used to quantitate hydrogen gas or, alternatively, may be used to assess hydrogen gas purity. For example, as explained above, where hydrogen gas sensor 11 is to be used for hydrogen gas quantitation, working electrode 21 may be held at an anodic potential to oxidize hydrogen gas that is present, converting the hydrogen gas into protons. The thus-generated protons may then be exchanged across proton exchange membrane 51 to first counter electrode 25, where such protons may be stored or intercalated. After a certain amount of time, first counter electrode 25 may be discharged, and protons previously stored therein may be conducted across proton exchange membrane 51 to second counter electrode 27, where they may be reduced. The oxidation current, in combination with the discharge profile, may then be used to determine the amount of hydrogen that was present. By contrast, where hydrogen gas sensor 11 is used to assess hydrogen purity assessment, the cell may be operated in a similar manner; however, in this case, only the oxidation current need be used to detect the presence of interferents.

As an illustration of the above, working electrode 21 may be initially biased such that it is held at anodically versus reference electrode 23, and first counter electrode 25 may be used as the counter electrode. The potential hold of working electrode 21 and first counter electrode 25 may be between 0.05 V to 0.7 V versus reference electrode 23. The oxidation current may be measured in this configuration for about 30 to 600 seconds. Following this period, first counter electrode 25 may be biased anodically versus reference electrode 23, and second counter electrode 27 may be used as the counter electrode. The discharge current profile may be measured in this configuration for about 60 to 900 seconds. The oxidation current and discharge current profile may then be used to determine either the concentration of hydrogen or its purity. If desired, cleaning pulses may be used to refresh the sensor surface by holding working electrode 21 at a potential of between 1 V to 2 V versus reference electrode 23.

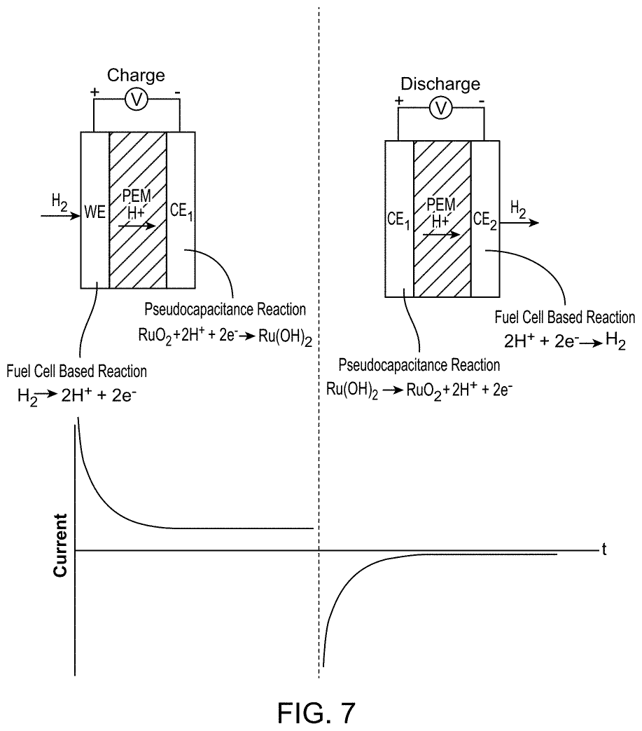

Referring now to FIG. 7, there is schematically shown the mechanism by which a hydrogen gas sensor, like hydrogen gas sensor 11, may be used, for example, to quantitate hydrogen gas. As can be seen, the sensor cell may be held so that hydrogen gas is oxidized at the working electrode (WE) held anodically and is conducted across the proton exchange membrane (PEM) to the ruthenium oxide first counter electrode (CE₁), where it is stored in a pseudocapacitance type reaction. After a certain amount of time, the cell is discharged where the first counter electrode (CE₁) is held anodically, protons reform, and are conducted across the proton exchange membrane (PEM) from the first counter electrode (CE₁) to the second counter electrode (CE₂), where they are reduced back to hydrogen (or form water in the presence of oxygen).

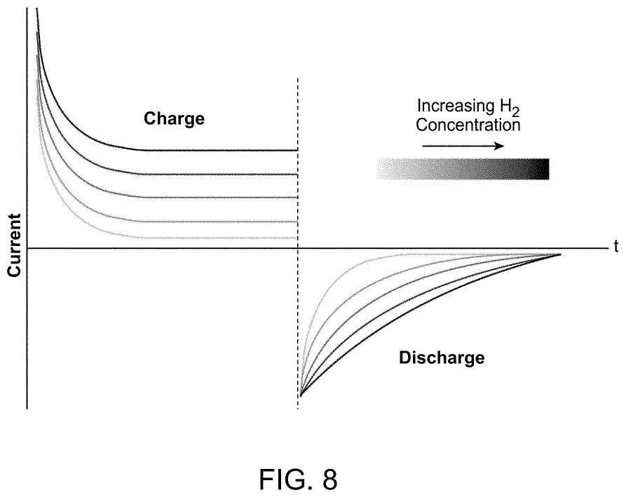

As can be seen in FIG. 8, when using the hydrogen gas sensor of the present invention for hydrogen quantification, the faradaic component of the oxidation current is proportional to the hydrogen concentration during charge operation. The amount of total charge passed over time is also proportional to hydrogen levels throughout the charging operation. Working electrode 21 and second counter electrode 27 would have both faradaic and capacitive currents, whereas first counter electrode 25 would be capacitive. The current profile would be the cell current between these electrodes, so it would be more of a mixed phenomenon. The overall profiles would be the same as what is presented in FIG. 8, assuming that first counter electrode 25 does not become saturated during the charging step nor are there appreciable reactions occurring on first counter electrode 25.

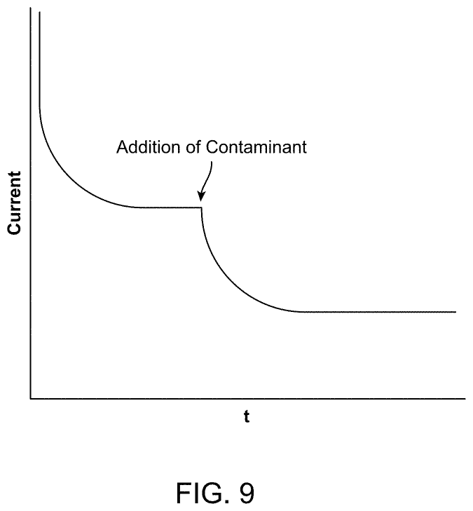

By contrast, as can be seen in FIG. 9, when using the hydrogen gas sensor of the present invention for assessing hydrogen gas purity, a steady state faradaic current may be obtained in pure hydrogen conditions; however, upon exposure to contaminants, the current drops. This drop in current value may then be used to determine the concentration of contaminant present.

The techniques described above for quantitating hydrogen gas or for assessing hydrogen gas purity may be performed using electronics connected to hydrogen gas sensor 11 through its contact pad region 39. Such electronics may include a potentiostat to control the potential bias and to measure an output current response. Such electronics may be programmed with internal calibration curves that may be used to relate measured oxidation currents and/or discharge profiles to a concentration of hydrogen gas or to potential interferent species. The raw data and calculated readings may be stored locally or may be wirelessly transmitted to an end user.

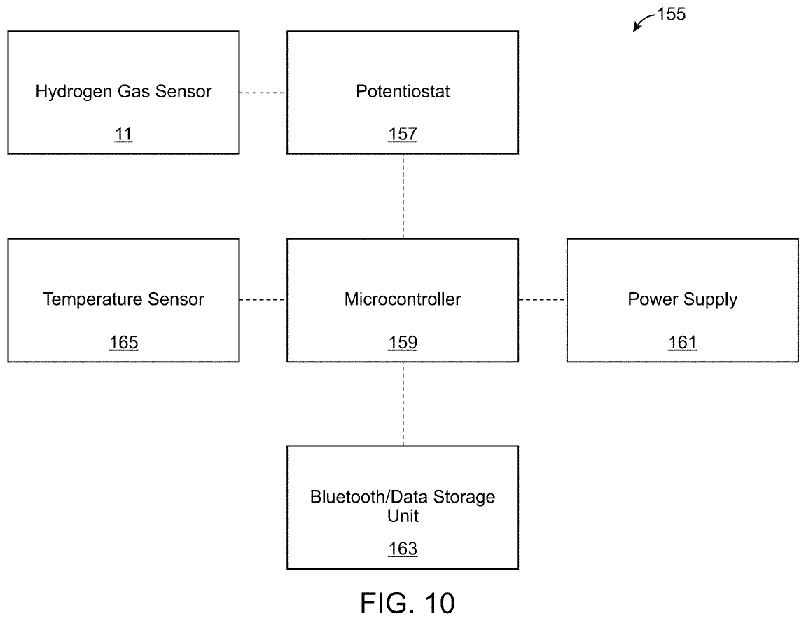

Referring now to FIG. 10, there is shown a simplified schematic diagram of a first embodiment of a hydrogen gas sensor system constructed according to the present invention, the hydrogen gas sensor system being represented generally by reference numeral 155. Details of hydrogen gas sensor system 155 that are discussed elsewhere in this application or that are not critical to an understanding of the invention may be omitted from FIG. 10 and/or from the accompanying description herein or may be shown in FIG. 10 and/or described herein in a simplified manner.

Hydrogen gas sensor system 155, which may be used in the ways described above to quantitate hydrogen gas and/or to assess hydrogen gas purity, may comprise hydrogen gas sensor 11.

Hydrogen gas sensor system 155 may further comprise a potentiostat 157. Potentiostat 157 may be operatively coupled to working electrode 21, reference electrode 23, first counter electrode 25, and second counter electrode 27, of hydrogen gas sensor 11 to apply any necessary voltages and to measure any currents generated.

Hydrogen gas sensor system 155 may further comprise a microcontroller 159. Microcontroller 159 may be operatively coupled to potentiostat 157 to control the operation of potentiostat 157 and to record any currents measured by potentiostat 157, as well as to compare any of the measurements made by potentiostat 157 to appropriate standards. Microcontroller 159 may also be used for any time measurements.

Hydrogen gas sensor system 155 may further comprise a power supply 161. Power supply 161 may be operatively coupled to microcontroller 159 to provide power to microcontroller 159.

Hydrogen gas sensor system 155 may further comprise a Bluetooth/data storage unit 163. Bluetooth/data storage unit 163 may be operatively coupled to microcontroller 159 to store the results of any testing, as well as to store standards that may be used in comparison with measurement data.

Hydrogen gas sensor system 155 may further comprise a temperature sensor 165, which is operatively coupled to microcontroller 159. Temperature sensor 165 may be used to obtain temperature measurements so that, if hydrogen gas sensor 11 was calibrated at a specific temperature, but the measurements are taken at another temperature, appropriate adjustments may be made.

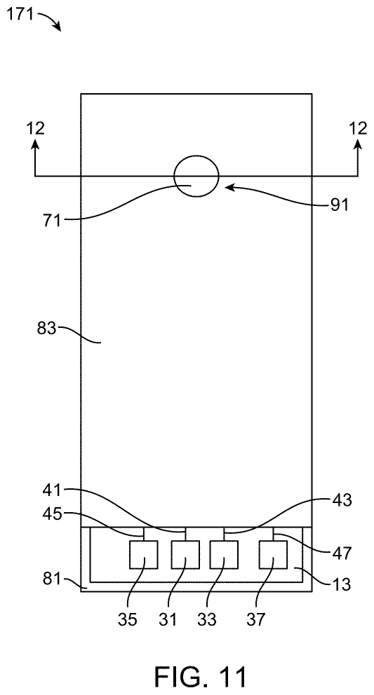

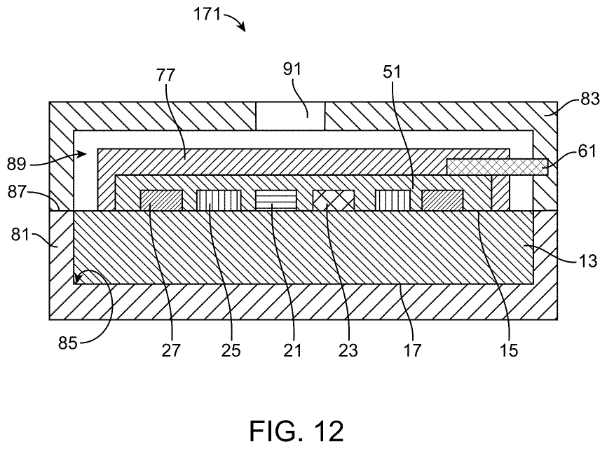

Referring now to FIGS. 11 and 12, there are shown various views of a second embodiment of a hydrogen gas sensor constructed according to the present invention, the hydrogen gas sensor being represented generally by reference numeral 171. Details of hydrogen gas sensor 171 that are discussed elsewhere in this application or that are not critical to an understanding of the invention may be omitted from one or more of FIGS. 11 and 12 and/or from the accompanying description herein or may be shown in one or more of FIGS. 11 and 12 and/or described herein in a simplified manner.

Hydrogen gas sensor 171 may be similar in most respects to hydrogen gas sensor 11. In fact, the principal difference between the two hydrogen gas sensors may be that, whereas hydrogen gas sensor 11 may include a permselective coating 71, hydrogen gas sensor 171 may omit permselective coating 71.

Hydrogen gas sensor 171 may be used in a similar fashion to hydrogen gas sensor 11 and may be used in the various manners discussed above for hydrogen gas sensor 11. As in the case of hydrogen gas sensor 11, for certain applications, second counter electrode 27 of hydrogen gas sensor 171 may be omitted.

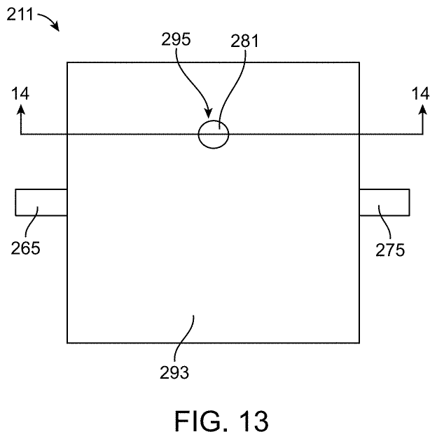

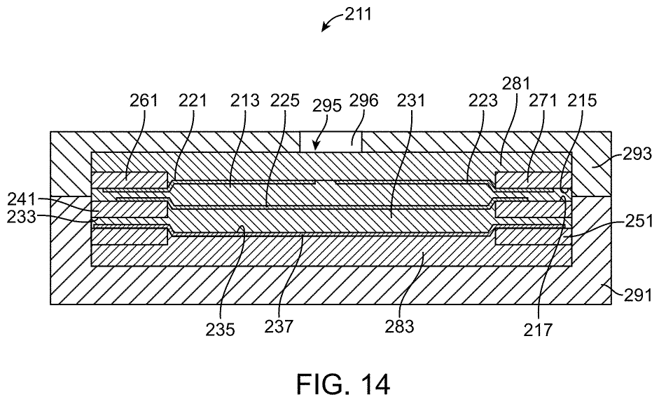

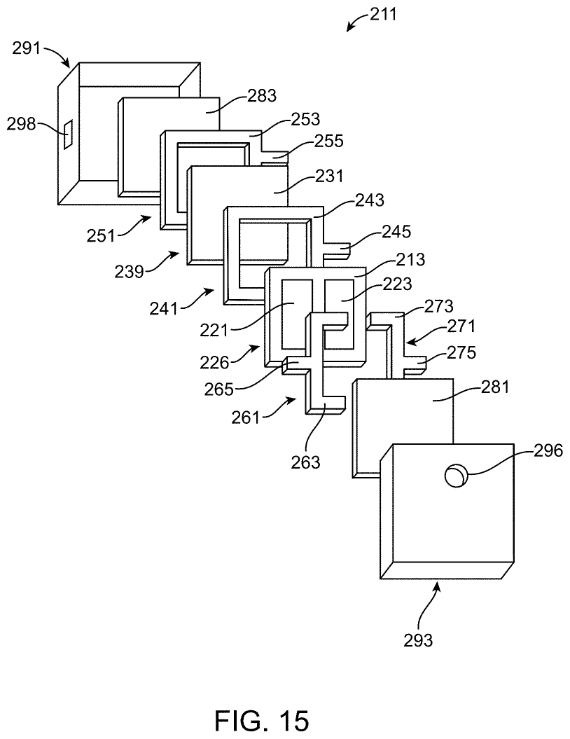

Referring now to FIGS. 13 through 15, there are shown various views of a third embodiment of a hydrogen gas sensor constructed according to the present invention, the hydrogen gas sensor being represented generally by reference numeral 211. Details of hydrogen gas sensor 211 that are discussed elsewhere in this application or that are not critical to an understanding of the invention may be omitted from one or more of FIGS. 13 through 15 and/or from the accompanying description herein or may be shown in one or more of FIGS. 13 through 15 and/or described herein in a simplified manner.

Hydrogen gas sensor 211 may comprise a first proton exchange membrane 213. First proton exchange membrane 213 may be in the form of a thin film membrane having a top surface 215 and a bottom surface 217. First proton exchange membrane 213 may consist of or comprise any of the materials described above in connection with proton exchange membrane 51 of hydrogen gas sensor 11. In the present embodiment, first proton exchange membrane 213 is shown having a generally rectangular (e.g., square) surface area or footprint; however, it is to be understood that the shape of first proton exchange membrane 213 is merely exemplary and that first proton exchange membrane 213 could have a different shape.

Hydrogen gas sensor 211 may further comprise a working electrode 221, a reference electrode 223, and a first counter electrode 225. In the present embodiment, working electrode 221 and reference electrode 223 may be spaced apart from one another, each may have a generally rectangular shape, and each may be applied directly to top surface 215 of first proton exchange membrane 213 while being spaced inwardly from the periphery thereof. Working electrode 221 may be similar in composition to working electrode 21 of hydrogen gas sensor 11, and reference electrode 223 may be similar in composition to reference electrode 23 of hydrogen gas sensor 11. First counter electrode 225, which may be applied directly to bottom surface 217 of first proton exchange membrane 213, may be similar in composition to first counter electrode 25 of hydrogen gas sensor 11 and may have a generally rectangular shape, first counter electrode 225 preferably being centered on bottom surface 217.

Working electrode 221 and reference electrode 223 may have similar surface areas to one another. By contrast, the surface area of first counter electrode 225 may be greater than that of each of working electrode 221 and reference electrode 223. In fact, the surface area of first counter electrode 225 may be similar to the combined surface areas of working electrode 221 and reference electrode 223. It should be noted that the shapes, sizes and placement of working electrode 221, reference electrode 223, and first counter electrode 225 in the present embodiment are merely exemplary.

The combination of first proton exchange membrane 213, working electrode 221, reference electrode 223, and first counter electrode 225 may be referred to herein as a first membrane electrode assembly (MEA) 226. First membrane electrode assembly 226 may be constructed using conventional MEA fabrication techniques, such as, but not limited to, coating the respective electrode materials onto the proton exchange membrane material using spray coating or roll coating. More specifically, membrane electrode assembly 226 may be fabricated by coating the respective electrode materials onto transfer sheets and then pressing the transfer sheets against first proton exchange membrane 213.

Hydrogen gas sensor 211 may further comprise a second proton exchange membrane 231. In the present embodiment, second proton exchange membrane 231 may be (but need not be) similar in size, shape and composition to first proton exchange membrane 213 and may be in the form of a thin film membrane having a top surface 233 and a bottom surface 235.

Hydrogen gas sensor 211 may further comprise a second counter electrode 237. In the present embodiment, second counter electrode 237 may have a generally rectangular shape and may be applied directly to bottom surface 235 of second proton exchange membrane 231, with second counter electrode 237 being centered on bottom surface 235. It should be noted that the shape of second counter electrode 237 in the present embodiment is merely exemplary. Second counter electrode 237 may be similar in composition to second counter electrode 27 of hydrogen gas sensor 11. Second counter electrode 237 may have a surface area that is similar to, or greater than, that of first counter electrode 225.

The combination of second proton exchange membrane 231 and second counter electrode 237 may be referred to herein as a second membrane electrode assembly (MEA) 239. Second membrane electrode assembly 239 may be constructed by applying the appropriate electrode material to the proton exchange membrane in a fashion similar to that described above in connection with first membrane electrode assembly 226.

Hydrogen gas sensor 211 may further comprise a first current collector 241, which may be positioned between and in direct contact with each of the bottom of first membrane electrode assembly 226 and the top of second membrane electrode assembly 239. First current collector 241 may be a unitary (i.e., one-piece) structure made of an electrically-conductive material, such as a suitable metal. First current collector 241 may be shaped to include a main portion 243 and a tab 245. Main portion 243, which may have a generally rectangular frame-like shape, may be appropriately dimensioned and positioned so that, with first current collector 241 in direct contact with bottom surface 217 of first proton exchange membrane 213, main portion 243 may be in direct contact with first counter electrode 225. The outer dimensions of main portion 243 may be similar to the outer dimensions of first proton exchange membrane 213. Tab 245, which extends outwardly from main portion 243, may be used to mount one end of a first electrical lead (not shown).

Hydrogen gas sensor 211 may further comprise a second current collector 251, which may be in direct contact with the bottom of second membrane electrode assembly 239. Second current collector 251 may be similar in size, shape and composition to first current collector 241 and may be shaped to include a main portion 253 and a tab 255. Main portion 253, which may have a generally rectangular frame-like shape, may be appropriately dimensioned and positioned so that, with second current collector 251 in direct contact with bottom surface 235 of second proton exchange membrane 231, main portion 253 may be in direct contact with second counter electrode 237. Tab 255, which extends outwardly from main portion 253, may be used to mount one end of a second electrical lead (not shown).

Hydrogen gas sensor 211 may further comprise a third current collector 261, which may be in direct contact with the top of first membrane electrode assembly 226. Third current collector 261, which may be similar in composition to first current collector 241, may be shaped to include a main portion 263 and a tab 265. Main portion 263 may be generally C-shaped and may be appropriately dimensioned and positioned so that, with third current collector 261 in direct contact with top surface 215 of first proton exchange membrane 213, main portion 263 may be in direct contact with working electrode 221. Tab 265, which extends outwardly from main portion 263, may be used to mount one end of a third electrical lead (not shown).

Hydrogen gas sensor 211 may further comprise a fourth current collector 271, which may also be in direct contact with the top of first membrane electrode assembly 226. Fourth current collector 271, which may be a mirror-image of third current collector 261 and which may be identical in composition thereto, may be shaped to include a main portion 273 and a tab 275. Main portion 273 may be appropriately dimensioned and positioned so that, with fourth current collector 271 in direct contact with top surface 215 of first proton exchange membrane 213, main portion 273 may be in direct contact with reference electrode 223. Main portion 273 of fourth current collector 271 and main portion 263 of third current collector 261 may collectively be similar in size and shape to each of main portion 243 of first current collector 241 and main portion 253 of second current collector 251. Tab 275, which extends outwardly from main portion 273, may be used to mount one end of a fourth electrical lead (not shown).

Hydrogen gas sensor 211 may further comprise a first protective barrier 281 and a second protective barrier 283. First protective barrier 281 and second protective barrier 283 may be identical to one another in size, shape, and composition and may be similar in composition to protective barrier 77 of hydrogen gas sensor 11. First protective barrier 281 may be positioned directly over third current collector 261 and fourth current collector 271 and may be appropriately dimensioned to cover the top surface of main portion 263 of third current collector 261 and the top surface of main portion 273 of fourth current collector 271. Second protective barrier 283 may be positioned directly under second current collector 251 and may be appropriately dimensioned to cover the bottom surface of main portion 253 of second current collector 251.

Hydrogen gas sensor 211 may further comprise a housing. In the present embodiment, the housing may comprise a bottom portion 291 and a top portion 293, wherein bottom portion 291 and top portion 293 may collectively define a cavity 295 appropriately dimensioned to receive the above-described components of hydrogen gas sensor 211. Each of bottom portion 291 and top portion 293 may be a unitary (i.e., one-piece) structure, which may be made by injection molding and which may consist of or comprise a suitable plastic material, such as, but not limited to, an acrylic, an acrylonitrile butadiene styrene, a nylon, a polycarbonate, a polyethylene, a polypropylene, a polystyrene, etc. Top portion 293 may be removably or permanently joined to bottom portion 291. Top portion 293 may be shaped to include an aperture 296 to allow gas to enter cavity 295 for analysis. Bottom portion 291 may have a plurality of openings 298 (only one of which is shown), through which tabs 265, 275, 245 and 255, respectively, may be inserted.

Preferably, bottom portion 291 and top portion 293 are appropriately dimensioned to keep the components disposed with cavity 295 under appropriate pressure to maintain contact between adjacent components. For example, when assembled, first protective barrier 281 may be in direct contact with each of third current collector 261, fourth current collector 271, working electrode 221, reference electrode 223, and an exposed portion of top surface 215 of proton exchange membrane 213; third current collector 261 may be in direct contact with working electrode 221; fourth current collector 271 may be in direct contact with reference electrode 223; first counter electrode 225 may be in direct contact with top surface 233 of proton exchange membrane 231; first current collector 241 may be in direct contact with each of first counter electrode 225 and top surface 233 of proton exchange membrane 231; second protective barrier 283 may be in direct contact with each of second counter electrode 227 and second current collector 251; and second current collector 251 may be in direct contact with second counter electrode 227.

Although not shown, hydrogen gas sensor 211 may further include one or more sorbent materials, which may be saturated with water, for use in keeping hydrated first proton exchange membrane 213 and/or second proton exchange membrane 231. Additionally and/or alternatively, although not shown, hydrogen gas sensor 211 may further include one or more permselective coatings like permselective coating 71. Additionally and/or alternatively, although not shown, hydrogen gas sensor 211 may further include one or more gaskets or other hardware that may be used, for example, to seal the electronic components from moisture and/or to prevent moisture from escaping any sorbent materials. Such hardware may seal the sensor so that only a small aperture exposes the sensor and is in direct contact with the protective barrier layer. The hardware also may seal the sorbent material as to ensure membrane hydration and prevent moisture from reaching the board electronics. Said hardware may also be designed in a form factor where it can be easily mounted to surfaces.

As can be appreciated, in certain applications, such as where hydrogen gas purity is being assessed in the manner described above, a second counter electrode is not essential; consequently, for such applications, hydrogen gas sensor 211 could be modified to omit second polymer exchange membrane 231, second counter electrode 237, and second current collector 251.

Hydrogen gas sensor 211 may be used in any of the ways discussed above in connection with hydrogen gas sensor 11.

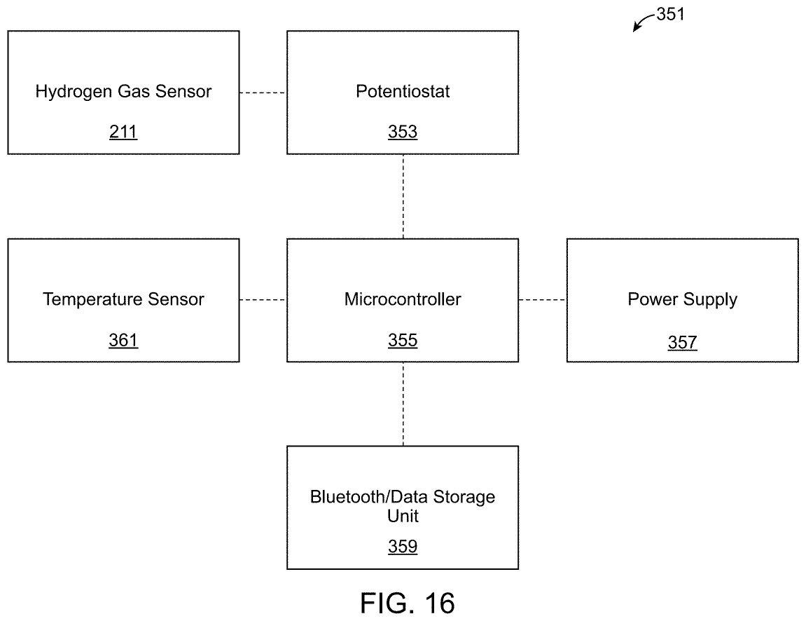

Referring now to FIG. 16, there is shown a simplified schematic diagram of a second embodiment of a hydrogen gas sensor system constructed according to the present invention, the hydrogen gas sensor system being represented generally by reference numeral 351. Details of hydrogen gas sensor system 351 that are discussed elsewhere in this application or that are not critical to an understanding of the invention may be omitted from FIG. 16 and/or from the accompanying description herein or may be shown in FIG. 16 and/or described herein in a simplified manner.

Hydrogen gas sensor system 351, which may be used in the ways described above to quantitate hydrogen gas and/or to assess hydrogen gas purity, may comprise hydrogen gas sensor 211.

Hydrogen gas sensor system 351 may further comprise a potentiostat 353. Potentiostat 353 may be operatively coupled to working electrode 221, reference electrode 223, first counter electrode 225, and second counter electrode 227, of hydrogen gas sensor 211 to apply any necessary voltages and to measure any currents generated.

Hydrogen gas sensor system 351 may further comprise a microcontroller 355.

Microcontroller 355 may be operatively coupled to potentiostat 353 to control the operation of potentiostat 353 and to record any currents measured by potentiostat 353, as well as to compare any of the measurements made by potentiostat 353 to appropriate standards. Microcontroller 355 may also be used for any time measurements.

Hydrogen gas sensor system 351 may further comprise a power supply 357. Power supply 357 may be operatively coupled to microcontroller 355 to provide power to microcontroller 355.

Hydrogen gas sensor system 351 may further comprise a Bluetooth/data storage unit 359. Bluetooth/data storage unit 359 may be operatively coupled to microcontroller 355 to store the results of any testing, as well as to store standards that may be used in comparison with measurement data.

Hydrogen gas sensor system 351 may further comprise a temperature sensor 361, which is operatively coupled to microcontroller 355. Temperature sensor 361 may be used to obtain temperature measurements so that, if hydrogen gas sensor 211 was calibrated at a specific temperature, but the measurements are taken at another temperature, appropriate adjustments may be made.

The electrodes of the hydrogen gas sensors discussed above may be reduced in size as compared to those of traditional hydrogen gas sensors of the type having a fuel cell-type construction. For example, such electrodes may be of microscale dimension while still maintaining the same or similar relative surface area ratios. Smaller electrode dimensions may be advantageous in reducing the response time of the sensor as faradaic steady states may be reached in a faster manner as electrode size is decreased. Additionally, sensitivity may be improved by using microelectrodes. Where the sensor of the present invention is used for hydrogen gas quantitation, microelectrodes structures, such as interdigitation, may provide enhanced current profile with reduced limitations on ionic transport. Where the sensor of the present invention is used for hydrogen gas purity assessment, the catalyst may become poisoned in a faster time scale; consequently, current drops may be noticeable at smaller concentrations in faster timescales. Membrane hydration may also be mitigated by making the proton exchange membrane much larger in size relative to the electrode. Electroosmotic water loss may be proportional to the electrode size; thus, reducing the size may improve issues related to water loss.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A hydrogen gas sensor, the hydrogen gas sensor comprising:
(a) a housing, the housing including a cavity and an aperture, the aperture permitting gas from outside the housing to enter the cavity;
(b) a first proton exchange membrane, the first proton exchange membrane being disposed within the cavity;
(c) a working electrode, the working electrode being disposed within the cavity and coupled to the first proton exchange membrane;
(d) a reference electrode, the reference electrode being disposed within the cavity and coupled to the first proton exchange membrane; and
(e) a first counter electrode, the first counter electrode being disposed within the cavity and coupled to the first proton exchange membrane, wherein the first counter electrode comprises one or more materials with pseudo-capacitor characteristics capable of proton intercalation.

2. The hydrogen gas sensor as claimed in claim 1 wherein the one or more materials with pseudo-capacitor characteristics capable of proton intercalation is at least one member selected from the group consisting of transition metal oxides, transition metal sulfides, and electron-conducting polymers.

3. The hydrogen gas sensor as claimed in claim 1 wherein the one or more materials with pseudo-capacitor characteristics capable of proton intercalation is at least one member selected from the group consisting of ruthenium oxide, tungsten oxide, titanium oxide, vanadium oxide, iridium oxide, iron oxide, manganese oxide, and titanium sulfide.

4. The hydrogen gas sensor as claimed in claim 1 wherein the one or more materials with pseudo-capacitor characteristics capable of proton intercalation comprises ruthenium oxide.

5. The hydrogen gas sensor as claimed in claim 1 wherein the working electrode has a working electrode surface area, wherein the first counter electrode has a first counter electrode surface area, and wherein the first counter electrode surface area is greater than the working electrode surface area.

6. The hydrogen gas sensor as claimed in claim 1 wherein the first counter electrode surface area is at least about twice the working electrode surface area.

7. The hydrogen gas sensor as claimed in claim 1 further comprising a second counter electrode, the second counter electrode being disposed within the cavity and coupled to the first proton exchange membrane.

8. The hydrogen gas sensor as claimed in claim 7 wherein the second counter electrode has a second counter electrode surface area and wherein the second counter electrode surface area is greater than the first counter electrode surface area.

9. The hydrogen gas sensor as claimed in claim 7 wherein the working electrode has a working electrode surface area, wherein the reference electrode has a reference electrode surface area, wherein the first counter electrode has a first counter electrode surface area, wherein the second counter electrode has a second counter electrode surface area, wherein the reference electrode surface area is substantially equal to the working electrode surface area, wherein the first counter electrode surface area is at least about twice as great as each of the working electrode surface area and the reference electrode surface area individually, and wherein the second counter electrode surface area is greater than the first counter electrode surface area.

10. The hydrogen gas sensor as claimed in claim 7 wherein each of the working electrode and the second counter electrode comprises one or more noble metal electrocatalyst materials.

11. The hydrogen gas sensor as claimed in claim 10 wherein the one or more noble metal electrocatalyst materials is at least one member selected from the group consisting of platinum, palladium, gold, and alloys thereof.

12. The hydrogen gas sensor as claimed in claim 1 wherein the reference electrode comprises one or more pseudo-reference electrode materials.

13. The hydrogen gas sensor as claimed in claim 12 wherein the one or more pseudo-reference electrode materials is at least one member selected from the group consisting of silver, a silver halide, gold, platinum, and platinum black.

14. The hydrogen gas sensor as claimed in claim 1 further comprising a substrate, the substrate comprising opposing top and bottom surfaces, wherein each of the working electrode, the reference electrode, and the first counter electrode is disposed over the top surface of the substrate, and wherein at least a portion of the first proton exchange membrane is disposed over and in direct contact with each of the working electrode, the reference electrode, and the first counter electrode.

15. The hydrogen gas sensor as claimed in claim 14 wherein the substrate is made of one or more electrically non-conductive, chemically inert materials.

16. The hydrogen gas sensor as claimed in claim 14 further comprising a second counter electrode, wherein the second counter electrode is disposed over the top surface of the substrate, and wherein at least a portion of the first proton exchange membrane is disposed over and in direct contact with the second counter electrode.

17. The hydrogen gas sensor as claimed in claim 16 further comprising a first contact pad, a second contact pad, a third contact pad, and a fourth contact pad, wherein the first contact pad is disposed on the substrate outside the cavity and is electrically coupled to the working electrode by a first trace, wherein the second contact pad is disposed on the substrate outside the cavity and is electrically coupled to the reference electrode by a second trace, wherein the third contact pad is disposed on the substrate outside the cavity and is electrically coupled to the first counter electrode by a third trace, and wherein the fourth contact pad is disposed on the substrate outside the cavity and is electrically coupled to the second counter electrode by a fourth trace.

18. The hydrogen gas sensor as claimed in claim 17 further comprising a dielectric film, the dielectric film positioned over at least a portion of each of the first trace, the second trace, the third trace, and the fourth trace.

19. The hydrogen gas sensor as claimed in claim 14 further comprising a permselective coating, the permselective coating being disposed on the first proton exchange membrane to inhibit interfering gas species from reaching one or more of the working electrode, the reference electrode, and the first counter electrode.

20. The hydrogen gas sensor as claimed in claim 19 wherein the permselective coating has a thickness of about 100 to 1000 microns and comprises at least one material selected from the group consisting of polymethylmethacrylate, fluorinated ethylene propylene, polyaniline, polytetrafluoroethylene (PTFE), and polyvinylidene fluoride (PVDF).

21. The hydrogen gas sensor as claimed in claim 1 wherein the first proton exchange membrane comprises a perfluorosulfonic acid polymer.

22. The hydrogen gas sensor as claimed in claim 21 wherein the first proton exchange membrane has a thickness of about 50 to 500 microns.

23. The hydrogen gas sensor as claimed in claim 1 further comprising a sorbent material containing water for use in keeping the first proton exchange membrane hydrated, the sorbent material being disposed within the cavity and coupled to the first proton exchange membrane.

24. The hydrogen gas sensor as claimed in claim 1 further comprising a protective barrier, the protective barrier being positioned in the cavity to block particulate matter and water from reaching at least one of the working electrode, the reference electrode, and the first counter electrode.

25. The hydrogen gas sensor as claimed in claim 24 wherein the protective barrier comprises at least one gas permeable material selected from the group consisting of a porous polytetrafluoroethylene (PTFE), carbon paper, carbon fiber paper, and silicone.

26. The hydrogen gas sensor as claimed in claim 1 wherein the first proton exchange membrane has opposing first and second surfaces, wherein the working electrode has opposing first and second surfaces, wherein the first surface of the working electrode is positioned in direct contact with the first surface of the first proton exchange membrane, and wherein the first surface of the first counter electrode is positioned in direct contact with the second surface of the first proton exchange membrane.

27. The hydrogen gas sensor as claimed in claim 26 wherein the reference electrode has opposing first and second surfaces and wherein the first surface of the reference electrode is positioned in direct contact with the first surface of the first proton exchange membrane.

28. The hydrogen gas sensor as claimed in claim 26 further comprising a second proton exchange membrane, wherein the second proton exchange membrane is disposed within the cavity, wherein the second proton exchange membrane has opposing first and second surfaces, and wherein the second surface of the first counter electrode is in direct contact with the first surface of the second polymer exchange membrane.

29. The hydrogen gas sensor as claimed in claim 28 further comprising a second counter electrode, wherein the second counter electrode is disposed within the cavity, wherein the second counter electrode has opposing first and second surfaces, and wherein the first surface of the second counter electrode is positioned in direct contact with the second surface of the second proton exchange membrane.

30. The hydrogen gas sensor as claimed in claim 29 further comprising a first current collector, a second current collector, a third current collector, and a fourth current collector, wherein the first current collector is positioned between the first proton exchange membrane and the second proton exchange membrane and is electrically coupled to the first counter electrode, wherein the second current collector is positioned along the second surface of the second proton exchange membrane and is electrically coupled to the second counter electrode, wherein the third current collector is positioned along the first surface of the first proton exchange membrane and is electrically coupled to the working electrode, and wherein the fourth current collector is positioned along the first proton exchange membrane and is electrically coupled to the reference electrode.

31. The hydrogen gas sensor as claimed in claim 30 further comprising a first protective barrier and a second protective barrier, wherein the first protective barrier is positioned outside the third and fourth current collectors to block particulate matter and water from reaching the working electrode and the reference electrode, and wherein the second protective barrier is positioned outside the second current collector to block particulate matter and water from reaching the second counter electrode.

32. A method for assessing hydrogen gas purity, the method comprising the steps of:
(a) providing a hydrogen gas sensor, the hydrogen gas sensor comprising
(i) a proton exchange membrane,
(ii) a working electrode, the working electrode coupled to the proton exchange membrane,
(iii) a reference electrode, the reference electrode coupled to the proton exchange membrane, and
(iv) a first counter electrode, the first counter electrode comprising one or more materials with pseudo-capacitor characteristics capable of proton intercalation;
(b) applying a first potential difference between the working electrode and the reference electrode;
(c) exposing a hydrogen gas sample to the working electrode, whereby hydrogen gas is oxidized at the working electrode and protons travel from the working electrode to the first counter electrode via the proton exchange membrane and are stored in the first counter electrode;
(d) measuring an oxidation current as the hydrogen gas sample is oxidized; and
(e) comparing the measured oxidation current to standards to assess hydrogen gas purity.

33. The method as claimed in claim 32 further comprising, after step (d), applying a second potential difference between the working electrode and the reference electrode to strip any contaminants from the working electrode.

34. The method as claimed in claim 33 further comprising comparing the second potential difference used to strip the contaminants to standards to identify the contaminants.

35. The method as claimed in claim 32 wherein the one or more materials with pseudo-capacitor characteristics capable of proton intercalation is at least one member selected from the group consisting of transition metal oxides, transition metal sulfides, and electron-conducting polymers.

36. The method as claimed in claim 32 wherein the one or more materials with pseudo-capacitor characteristics capable of proton intercalation is at least one member selected from the group consisting of ruthenium oxide, tungsten oxide, titanium oxide, vanadium oxide, iridium oxide, iron oxide, manganese oxide, and titanium sulfide.

37. The method as claimed in claim 36 wherein the one or more materials with pseudo-capacitor characteristics capable of proton intercalation comprises ruthenium oxide.

38. The method as claimed in claim 32 wherein the working electrode has a working electrode surface area, wherein the first counter electrode has a first counter electrode surface area, and wherein the first counter electrode surface area is greater than the working electrode surface area.

39. The method as claimed in claim 38 wherein the first counter electrode surface area is at least about twice the working electrode surface area.

40. A method for quantitating hydrogen gas, the method comprising the steps of:
(a) providing a hydrogen gas sensor, the hydrogen gas sensor comprising
(i) a proton exchange membrane,
(ii) a working electrode, the working electrode coupled to the proton exchange membrane,
(iii) a reference electrode, the reference electrode coupled to the proton exchange membrane,
(iv) a first counter electrode, the first counter electrode coupled to the proton exchange membrane and comprising one or more materials with pseudo-capacitor characteristics capable of proton intercalation;
(v) a second counter electrode, the second counter electrode coupled to the proton exchange membrane;
(b) applying a potential difference between the working electrode and the reference electrode;
(c) exposing a sample to the working electrode, whereby hydrogen gas, if present, is oxidized at the working electrode to generate protons that travel from the working electrode to the first counter electrode via the proton exchange membrane and are intercalated in the first counter electrode;
(d) measuring an oxidation current for the sample; and
(e) comparing the measured oxidation current to standards to quantitate hydrogen gas.

41. The method as claimed in claim 40 further comprising, after step (d), the steps of:
applying a potential difference between the first counter electrode and the reference electrode to cause protons intercalated in the first counter electrode to be de-intercalated therefrom and to travel, via the proton exchange membrane, to the second counter electrode;
measuring a discharge current profile for the protons de-intercalated from the first counter electrode; and
comparing the discharge current profile to standards to quantitate hydrogen gas.

42. The method as claimed in claim 40 wherein the one or more materials with pseudo-capacitor characteristics capable of proton intercalation is at least one member selected from the group consisting of transition metal oxides, transition metal sulfides, and electron-conducting polymers.

43. The method as claimed in claim 42 wherein the one or more materials with pseudo-capacitor characteristics capable of proton intercalation is at least one member selected from the group consisting of ruthenium oxide, tungsten oxide, titanium oxide, vanadium oxide, iridium oxide, iron oxide, manganese oxide, and titanium sulfide.

44. The method as claimed in claim 43 wherein the one or more materials with pseudo-capacitor characteristics capable of proton intercalation comprises ruthenium oxide.

45. The method as claimed in claim 40 wherein the working electrode has a working electrode surface area, wherein the first counter electrode has a first counter electrode surface area, and wherein the first counter electrode surface area is greater than the working electrode surface area.

46. The method as claimed in claim 45 wherein the first counter electrode surface area is at least about twice the working electrode surface area.

47. The method as claimed in claim 41 wherein the first counter electrode has a first counter electrode surface area, wherein the second counter electrode has a second counter electrode surface area, and wherein the second counter electrode surface area is greater than the first counter electrode surface area.

48. A method for quantitating hydrogen gas, the method comprising the steps of:
(a) providing a hydrogen gas sensor, the hydrogen gas sensor comprising
(ii) a proton exchange membrane,
(ii) a working electrode, the working electrode coupled to the proton exchange membrane,
(iii) a reference electrode, the reference electrode coupled to the proton exchange membrane,
(iv) a first counter electrode, the first counter electrode coupled to the proton exchange membrane and comprising one or more materials with pseudo-capacitor characteristics capable of proton intercalation;
(v) a second counter electrode, the second counter electrode coupled to the proton exchange membrane;
(b) applying a potential difference between the working electrode and the reference electrode;
(c) exposing a sample to the working electrode for a measured period of time, whereby hydrogen gas, if present, is oxidized at the working electrode to generate protons that travel from the working electrode to the first counter electrode via the proton exchange membrane and are intercalated in the first counter electrode;
(d) applying a potential difference between the first counter electrode and the reference electrode to cause protons intercalated in the first counter electrode to be de-intercalated therefrom and to travel, via the proton exchange membrane, to the second counter electrode; (e) measuring a discharge current profile for the protons de-intercalated from the first counter electrode; and (f) comparing the discharge current profile to standards to quantitate hydrogen gas.

49. The method as claimed in claim 48 wherein the one or more materials with pseudo-capacitor characteristics capable of proton intercalation is at least one member selected from the group consisting of transition metal oxides, transition metal sulfides, and electron-conducting polymers.

50. The method as claimed in claim 49 wherein the one or more materials with pseudo-capacitor characteristics capable of proton intercalation is at least one member selected from the group consisting of ruthenium oxide, tungsten oxide, titanium oxide, vanadium oxide, iridium oxide, iron oxide, manganese oxide, and titanium sulfide.

51. The method as claimed in claim 50 wherein the one or more materials with pseudo-capacitor characteristics capable of proton intercalation comprises ruthenium oxide.

52. The method as claimed in claim 48 wherein the working electrode has a working electrode surface area, wherein the first counter electrode has a first counter electrode surface area, and wherein the first counter electrode surface area is greater than the working electrode surface area.

53. The method as claimed in claim 52 wherein the first counter electrode surface area is at least about twice the working electrode surface area.

54. The method as claimed in claim 48 wherein the first counter electrode has a first counter electrode surface area, wherein the second counter electrode has a second counter electrode surface area, and wherein the second counter electrode surface area is greater than the first counter electrode surface area.
